(19) United States
(12) Patent Application Publication
(10) Pub. No.: US 2023/0368927 A1
(43) Pub. Date: Nov. 16, 2023

(54) INFORMATION PROCESSING APPARATUS, INFORMATION PROCESSING METHOD, AND STORAGE MEDIUM

(71) Applicant: NEC Corporation, Minato-ku, Tokyo (JP)

(72) Inventors: Takumi OTANI, Tokyo (JP); Takeshi SASAMOTO, Tokyo (JP); Junichi INOUE, Tokyo (JP)

(73) Assignee: NEC Corporation, Minato-ku, Tokyo (JP)

(21) Appl. No.: 18/030,432

(22) PCT Filed: Oct. 9, 2020
(86) PCT No.: PCT/JP2020/038387

§ 371 Date: Apr. 5, 2023

(51) Int. Cl. G16H 50/80; G06V 40/16; G06Q 50/26

(52) U.S. Cl. CPC G16H 50/80; G06Q 50/265; G06V 40/172

(57) ABSTRACT

An information processing apparatus according to the present invention includes: an identifying unit that identifies a person with fever at an airport; and a generating unit that generates statistical information related to the person with fever based on user information acquired from the person with fever.

### TECHNICAL FIELD

The present invention relates to an information processing apparatus, an information processing method, and a storage medium.

### BACKGROUND ART

Patent Literature 1 discloses a risk determination system in which an inspection terminal installed at a gate that controls the entry and exit of passengers. The inspection terminal of the risk determination system acquires information on the degree of risk that the user is suffering from a disease from a risk determination device based on location information and biometric information (body temperature, pulse rate, etc.) acquired from a wearable terminal worn by the passenger, and displays the acquired information on a screen.

### CITATION LIST

### Patent Literature

PTL 1: Japanese Patent Application Laid-Open No. 2016-195639

### SUMMARY OF INVENTION

### Technical Problem

According to the system described in Patent Literature 1, when a user (passenger) who has passed through a specific gate is in a fever state, the user can be detected as a person of high risk. However, the system was not intended to provide an overall fever situation concerning airport users.

Accordingly, the present invention has been made in view of such circumstances and intends to provide an information processing apparatus, an information processing method, and a storage medium that can detect the overall fever situation regarding airport users.

### Solution to Problem

According to one aspect of the present invention, provided is an information processing apparatus including: an identifying unit that identifies a person with fever at an airport; and a generating unit that generates statistical information related to the person with fever based on user information acquired from the person with fever.

According to another aspect of the present invention, provided is an information processing method including: identifying a person with fever at an airport; and generating statistical information related to the person with fever based on user information acquired from the person with fever.

According to yet another aspect of the present invention, provided is a storage medium storing a program that causes a computer to perform: identifying a person with fever at an airport; and generating statistical information related to the person with fever based on user information acquired from the person with fever.

### Advantageous Effects of Invention

According to the present invention, an information processing apparatus, an information processing method, and a storage medium that can detect the overall fever situation regarding airport users.

### BRIEF DESCRIPTION OF DRAWINGS

FIG. 1 is a schematic diagram illustrating an example of an overall configuration of an information processing system according to a first example embodiment.

FIG. 2 is a diagram illustrating an example of information stored in a token ID information DB according to the first example embodiment.

FIG. 3 is a diagram illustrating an example of information stored in a passage history information DB according to the first example embodiment.

FIG. 4 is a diagram illustrating an example of information stored in an operation information DB according to the first example embodiment.

FIG. 5 is a block diagram illustrating an example of a hardware configuration of a management server according to the first example embodiment.

FIG. 6 is a block diagram illustrating an example of a hardware configuration of a check-in terminal according to the first example embodiment.

FIG. 7 is a block diagram illustrating an example of a hardware configuration of an automatic baggage drop-off machine according to the first example embodiment.

FIG. 8 is a block diagram illustrating an example of a hardware configuration of a security inspection apparatus according to the first example embodiment.

FIG. 9 is a block diagram illustrating an example of a hardware configuration of an automated gate apparatus according to the first example embodiment.

FIG. 10 is a block diagram illustrating an example of a hardware configuration of a boarding gate apparatus according to the first example embodiment.

FIG. 11 is a block diagram illustrating an example of a hardware configuration of an administrator terminal according to the first example embodiment.

FIG. 12 is a sequence chart illustrating an example of a process in a check-in procedure performed by the information processing system according to the first example embodiment.

FIG. 13 is a diagram illustrating a state where a face image and a thermography image are captured at the check-in terminal according to the first example embodiment.

FIG. 14 is a sequence chart illustrating an example of a process in an automatic baggage drop-off procedure performed by the information processing system according to the first example embodiment.

FIG. 15 is a sequence chart illustrating an example of a process in a security inspection procedure performed by the information processing system according to the first example embodiment.

FIG. 16 is a sequence chart illustrating an example of a process in a departure inspection procedure performed by the information processing system according to the first example embodiment.

FIG. 17 is a sequence chart illustrating an example of a process in an identity verification procedure before boarding performed by the management server according to the first example embodiment.

FIG. 18 is a diagram illustrating an example of measurement history information on body surface temperatures according to the first example embodiment.

FIG. 19 is a flowchart illustrating an example of a statistical analysis process according to the first example embodiment.

FIG. 20 is a flowchart illustrating an example of an identification process for a person with fever according to the first example embodiment.

FIG. 21 is a sequence chart illustrating an example of the statistical analysis process according to the first example embodiment.

FIG. 22 is a diagram illustrating an example of a statistical analysis conditions input screen displayed on an administrator terminal according to the first example embodiment.

FIG. 23 is a diagram illustrating an example of a person-with-fever analysis result screen displayed on the administrator terminal according to the first example embodiment.

FIG. 24 is a diagram illustrating an example of a person-with-fever list screen displayed on the administrator terminal according to the first example embodiment.

FIG. 25 is a diagram illustrating an example of a person-with-fever detail information screen displayed on the administrator terminal according to the first example embodiment.

FIG. 26 is a schematic diagram illustrating an example of an overall configuration of an information processing system according to a second example embodiment.

FIG. 27 is a schematic diagram illustrating an example of an external view of an entry gate terminal and an exit gate terminal forming an automatic customs gate apparatus according to the second example embodiment.

FIG. 28A is a block diagram illustrating an example of a hardware configuration of the entry gate terminal of the automatic customs gate apparatus according to the second example embodiment.

FIG. 28B is a block diagram illustrating an example of a hardware configuration of the exit gate terminal of the automatic customs gate apparatus according to the second example embodiment.

FIG. 29 is a sequence chart illustrating an example of a data coordination process between two countries in the information processing system according to the second example embodiment.

FIG. 30 is a sequence chart illustrating an example of a process in an entry inspection procedure performed by the information processing system according to the second example embodiment.

FIG. 31 is a sequence chart illustrating an example of a process in a customs inspection procedure performed by the information processing system according to the second example embodiment.

FIG. 32 is a function block diagram of an information processing apparatus according to a third example embodiment.

### DESCRIPTION OF EMBODIMENTS

Exemplary example embodiments of the present invention will be described below with reference to the drawings. Throughout the drawings, similar elements or corresponding elements are labeled with the same references, and the description thereof may be omitted or simplified.

### First Example Embodiment

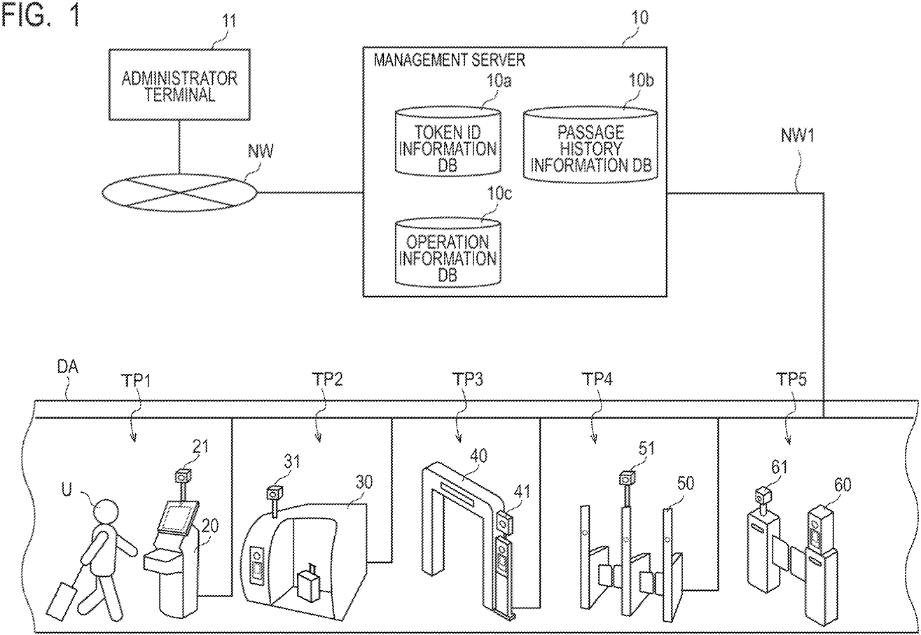

FIG. 1 is a schematic diagram illustrating an example of the overall configuration of an information processing system according to the present example embodiment. The information processing system according to the present example embodiment is a computer system that supports a series of procedures performed on a user U in a first country and a second country, respectively, when the user U departs from the first country at an airport DA of the first country and enters the second country at an airport of the second country by an airplane. The information processing system is run by a public institution such as an immigration control bureau or a trustee entrusted with the operation from such an institution, for example.

As illustrated in FIG. 1, the information processing system includes management servers 10, an administrator terminal 11, a check-in terminal 20, an automatic baggage drop-off machine 30, a security inspection apparatus 40, an automated gate apparatus 50, and a boarding gate apparatus 60. The management server 10 is connected to the administrator terminal 11 via network NW. The management server 10 is connected to the check-in terminal 20, the automatic baggage drop-off machine 30, the security inspection apparatus 40, the automated gate apparatus 50, and the boarding gate apparatus 60 in the airport DA via network NW1, respectively. The network NW and NW1, are each formed of a Wide Area Network (WAN) such as the Internet or a Local Area Network (LAN). The connection scheme may be a wireless scheme without being limited to a wired scheme.

The management servers 10 each are an information processing apparatus that manages various procedures on the user U during entry to or departure from countries. The management server 10 is installed in a facility of an airport company, an airline company, or the like, for example. Note that the management server 10 is not required to be a single server and may be configured as a server group including a plurality of servers. Further, the management server 10 is not necessarily required to be provided on a country basis and may be configured as a server used by a plurality of countries in a shared manner.

The management server 10 performs identity verification on the user U by matching a face image captured by the check-in terminal 20, which is a face authentication terminal, with a passport face image read from a passport by the check-in terminal 20.

Furthermore, the management server 10 performs identity verification on the user U by matching a face image captured by another face authentication terminal (each of the automatic baggage drop-off machine 30, the security inspection apparatus 40, the automated gate apparatus 50, the boarding gate apparatus 60, or the like) in the airport DA with a registered face image registered in a database, respectively.

Further, as illustrated in FIG. 1, the management server 10 includes a token ID information DB 10a, a passage history information DB 10b, and an operation information DB 10c. These databases are examples, and the management server 10 may further include other databases. Further, a plurality of databases may be aggregated into a single database.

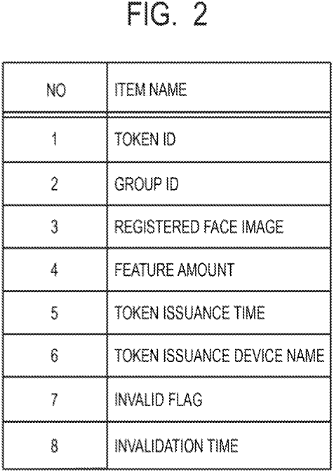

FIG. 2 is a diagram illustrating an example of information stored in the token ID information DB 10a. The token ID information DB 10a has data items of a token ID, a group ID, a registered face image, a feature amount, a token issuance time, a token issuance device name, an invalid flag, and an invalidation time.

The token ID is an identifier that uniquely identifies ID information. The token ID of the present example embodiment is issued by the management server 10 provided that a result of a matching process is that the matching is successful where the matching process is to match a captured face image, which is obtained by the user U capturing his/her face by himself/herself by using a face authentication terminal such as the check-in terminal 20, with a passport face image of the user U read from a passport by the face authentication terminal. Further, for example, after the user U finishes the travel from the first country to the second country, the token ID is invalidated. That is, a token ID is not an identifier used permanently but a onetime ID having a validity period (lifecycle).

Note that the term “matching is successful” in the present example embodiment means that a matching score indicating a similarity between biometric information on the user U and registered biometric information on a registrant is greater than or equal to a predetermined threshold. In contrast, the term “matching is unsuccessful” means that a matching score is less than the predetermined threshold.

The group ID is an identifier for grouping ID information. The registered face image is a face image registered for the user U. In the present example embodiment, a face image of the user U captured during the initial procedure in the airport DA of the first country or a passport face image read from an IC chip of a passport of the user U by a reading device is used as a registered face image stored in the token ID information DB 10a. The feature amount is a value extracted from biometric information (registered face image).

Further, although the term of biometric information in the present example embodiment means a face image and a feature amount extracted from the face image, the biometric information is not limited to a face image and a face feature amount. That is, biometric authentication may be performed by using an iris image, a fingerprint image, a palmprint image, an auricular image, or the like as biometric information on the user U.

The token issuance time is a time that the management server 10 issued a token ID. The token issuance device name is a device name from which a registered face image which triggered issuance of a token ID is acquired. The invalid flag is flag information indicating whether or not a token ID is currently valid. For example, upon issuance of a token ID, the invalid flag is set to a value indicating a state where the token ID is valid. Further, in response to satisfying a predetermined condition, the invalid flag is updated to a value indicating a state where a token ID is invalid. The invalidation time is a timestamp indicating a time the invalid flag is invalidated.

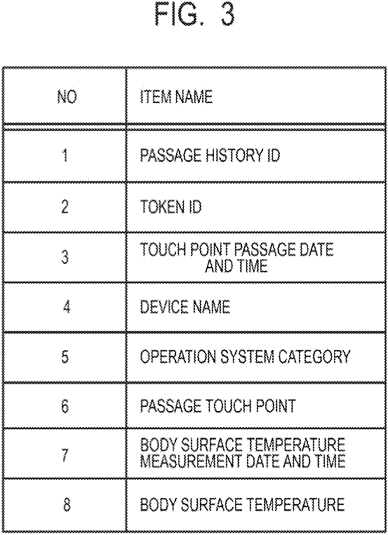

FIG. 3 is a diagram illustrating an example of information stored in the passage history information DB 10b. The passage history information DB 10b has data items of a passage history ID, a token ID, a touch point passage date and time, a device name, an operation system category, a passage touch point, a body surface temperature measurement date and time, and a body surface temperature. The passage history ID is an identifier that uniquely identifies passage history information. The touch point passage date and time is a timestamp indicating a time the user passes through a touch point. The device name is a machine name of an operation terminal used for a procedure at a touch point. The operation system category is a category of an operation system which an operation terminal belongs to. The passage touch point is a name of a touch point through which the user U passes. The body surface temperature measurement date and time is a timestamp when a body surface temperature of the user U is measured by capturing of a thermography image. The body surface temperature is a temperature measured for a skin surface of the user U.

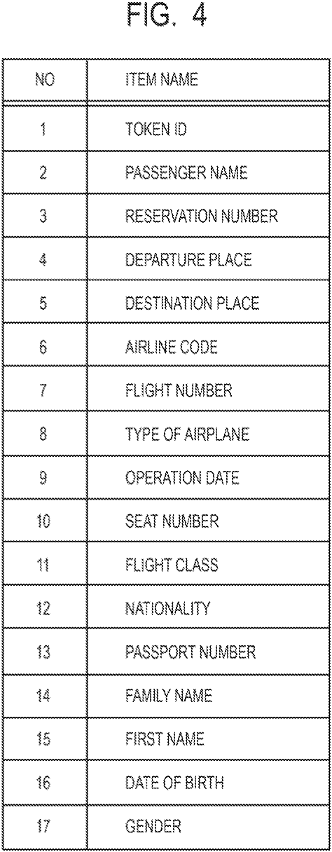

FIG. 4 is a diagram illustrating an example of information stored in the operation information DB 10c. The operation information DB 10c has data items of a token ID, a passenger name, a reservation number, a departure place, a destination place, an airline code, a flight number, a type of airplane, an operation date, a seat number, a flight class, a nationality, a passport number, a family name, a first name, a date of birth, and a gender.

The reservation number is an identifier that uniquely identifies boarding reservation information. The airline code is an identifier that uniquely identifies an airline company. The flight class is a class of a seat and may be, for example, first class, business class, economy class, or the like. In general, a seat of a higher flight class has a longer distance to the next seat and a longer distance (seat pitch) to the front and rear seats. Further, services that the user U may receive in an airport and a cabin are also different in accordance with a flight class.

Information on a passenger name, a reservation number, a departure place, a destination place, an airline code, a flight number, a type of an airplane, an operation date, a seat number, a nationality, a passport number, a family name, a first name, a date of birth, a gender, or the like may be acquired from a medium such as a passport and a boarding ticket or acquired from a database that manages reservation information (not illustrated) by using passport number, a reservation number, or the like as a key.

In such a way, the operation information DB 10c stores operation information about a predetermined operation in association with a token ID. In the present example embodiment, “predetermined operation” means a procedure operation (check-in / baggage drop-off / security inspection / departure inspection / identity verification on a passenger, or the like) performed at each touch point in an airport.

The administrator terminal 11 is installed at airport and airline facilities. The administrator terminal 11 is, for example, a terminal used by the administrator of the management server 10 for maintenance work and statistical analysis work. The administrator terminal 11 is, for example, a personal computer, a tablet terminal, etc.

Next, the apparatuses responsible for procedural operations on the user U in cooperation with the management server 10 in the airport DA according to the present example embodiment will be described.

The check-in terminal 20 is installed in a check-in lobby or a check-in counter in each of the airport DA. Hereafter, the procedural area where the check-in terminal 20 is installed is referred to as “touch point TP1”. The check-in terminal 20 is a self-service terminal operated by the user U by himself/herself to perform a check-in procedure (a boarding procedure). After completion of the check-in procedure at the touch point TP1, the user U proceeds to a baggage drop-off place or a security inspection site.

The automatic baggage drop-off machine 30 is installed in a region adjacent to a baggage counter (a manned counter) or a region near the check-in terminal 20 in each of the airport DA. Hereafter, the procedural area where the automatic baggage drop-off machine 30 is installed is referred to as “touch point TP2”. The automatic baggage drop-off machine 30 is a self-service terminal operated by the user U by himself/herself to perform a procedure to drop off, to an airline company, baggage not to be carried in the cabin. After completion of the baggage drop-off procedure at the touch point TP2, the user U proceeds to the security inspection site. When the user U does not drop off his/her baggage, the procedure at the touch point TP2 is omitted.

The security inspection apparatus 40 is installed in the security inspection site (hereafter, referred to as “touch point TP3”) in each of the airport DA. The term “security inspection apparatus” in the present example embodiment is used as a meaning including all of a metal detector that checks whether or not the user U is wearing a metal item that may be a dangerous object, an X-ray inspection device that uses an X-ray to check whether or not a dangerous object is included in carry-on baggage or the like, a passage control device that determines whether or not to permit passage of the user U at an entrance or an exit of the security inspection site, and the like. After completion of the security inspection procedure at the touch point TP3, the user U proceeds to a departure inspection site.

The automated gate apparatus 50 is installed at the departure inspection site (hereafter, referred to as “touch point TP4”) in each of the airport DA. The automated gate apparatus 50 is an apparatus that automatically performs a departure inspection procedure on the user U. After completion of the departure inspection procedure at the touch point TP4, the user U proceeds to a departure area where a duty-free shop and a boarding gate are provided.

The boarding gate apparatus 60 is installed to each boarding gate (hereafter, referred to as “touch point TP5”) in each of the airport DA. The boarding gate apparatus 60 is a passage control apparatus that checks whether or not the user U is a passenger of an airplane associated with the boarding gate. After completion of the procedure at the touch point TP5, the user U boards the airplane and departs to the second country. In such a way, the check-in terminal 20, the automatic baggage drop-off machine 30, the security inspection apparatus 40, the automated gate apparatus 50, and the boarding gate apparatus 60 are used when the user U departs from the first country.

Also, as illustrated in FIG. 1, the check-in terminal 20, the automatic baggage drop-off machine 30, the security inspection apparatus 40, the automated gate apparatus 50 and the boarding gate apparatus 60 include thermography devices 21, 31, 41, 51 and 61, respectively. Herein, the thermography device 21 is described as a representative example.

The thermography device 21 is an image capturing device that analyzes infrared rays emitted from an object and generates a thermography image representing a heat distribution. The thermography device 21 has the following advantages.

(A) It is possible to measure a body surface temperature in a contactless manner with an object to be measured.

(B) It is possible to visualize, as an image, a temperature distribution of a wide area as a plane rather than a temperature value at a point on an object.

(C) It is possible to measure a body surface temperature in real time because the response speed is high.

Because of such advantages, the thermography device 21 according to the present example embodiment is used for measuring the body surface temperature of the user U in the airport DA.

Next, a hardware configuration of devices forming the information processing system will be described. Note that, throughout a plurality of drawings, devices having the same name and differing only in the reference are devices having substantially the same function, and thus, the detailed description thereof will be omitted in the subsequent drawings.

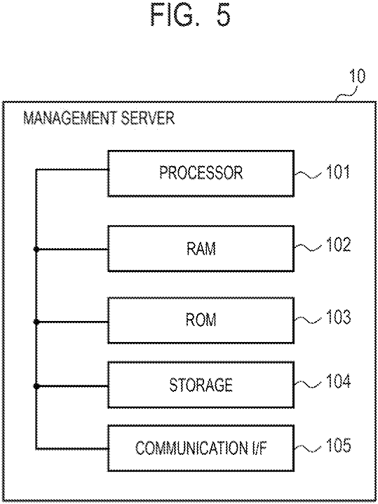

FIG. 5 is a block diagram illustrating an example of a hardware configuration of the management server 10. The management server 10 includes a processor 101, a random access memory (RAM) 102, a read only memory (ROM) 103, a storage 104, and a communication interface (I/F) 105, as a computer that performs calculation, control, and storage. These devices are connected to each other via a bus, a wiring, a drive device, or the like.

The processor 101 has functions of performing predetermined calculation in accordance with a program stored in the ROM 103, the storage 104, or the like and controlling each unit of the management server 10. Further, as the processor 101, one of a central processing unit (CPU), a graphics processing unit (GPU), a field programmable gate array (FPGA), a digital signal processor (DSP), and an application specific integrated circuit (ASIC) may be used, or a plurality thereof may be used in parallel.

The RAM 102 is formed of a volatile storage medium and provides a temporary memory area required for the operation of the processor 101. The ROM 103 is formed of a nonvolatile storage medium and stores information required such as a program used for the operation of the management server 10.

The storage 104 is formed of a nonvolatile storage medium and performs storage of a database, storage of an operating program of the management server 10, or the like. The storage 104 is formed of a hard disk drive (HDD) or a solid state drive (SSD), for example.

The communication I/F 105 is a communication interface based on a specification such as Ethernet (registered trademark), Wi-Fi (registered trademark), 4G, or the like and is a module for communicating with other devices.

The processor 101 loads a program stored in the ROM 103, the storage 104, or the like into the RAM 102 and executes the program to perform a predetermined calculation process. Further, the processor 101 controls each unit of the management server 10, such as the communication I/F 105, based on the program.

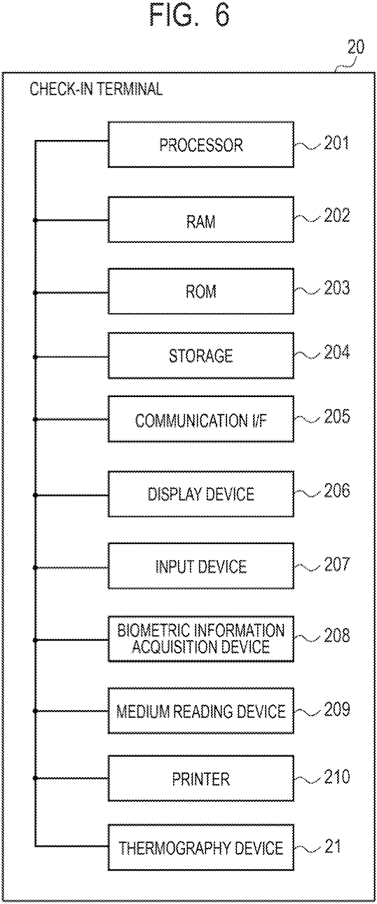

FIG. 6 is a block diagram illustrating an example of the hardware configuration of the check-in terminal 20. The check-in terminal 20 includes a processor 201, a RAM 202, a ROM 203, a storage 204, a communication I/F 205, a display device 206, an input device 207, a biometric information acquisition device 208, a medium reading device 209, a printer 210, and the thermography device 21. These devices are connected to each other via a bus, a wiring, a drive device, or the like.

The display device 206 is a liquid crystal display, an organic light emitting diode (OLED) display, or the like configured to display a moving image, a static image, a text, or the like and is used for presenting information to the user U.

The input device 207 is a keyboard, a pointing device, a button, or the like and accepts a user operation. The display device 206 and the input device 207 may be formed integrally as a touch panel.

The biometric information acquisition device 208 is a device that acquires a face image of the user U as biometric information on the user U. The biometric information acquisition device 208 is a digital camera having a Complementary Metal-Oxide-Semiconductor (CMOS) image sensor, a Charge Coupled Device (CCD) image sensor, or the like as a light receiving element, for example. The biometric information acquisition device 208 captures an image of a face of the user U standing in front of the device to acquire the face image, for example.

The medium reading device 209 is a device that reads information recorded or stored in a medium carried by the user U. The medium reading device 209 may be, for example, a code reader, an image scanner, a contactless integrated circuit (IC) reader, an optical character reader (OCR) device, or the like. Further, a recording medium or a storage medium may be, for example, a paper airline ticket, a mobile terminal displaying a receipt of an e-ticket, or the like. The printer 210 prints a boarding ticket in which boarding information and guidance information about procedures up to boarding are printed at the time of completion of a check-in procedure.

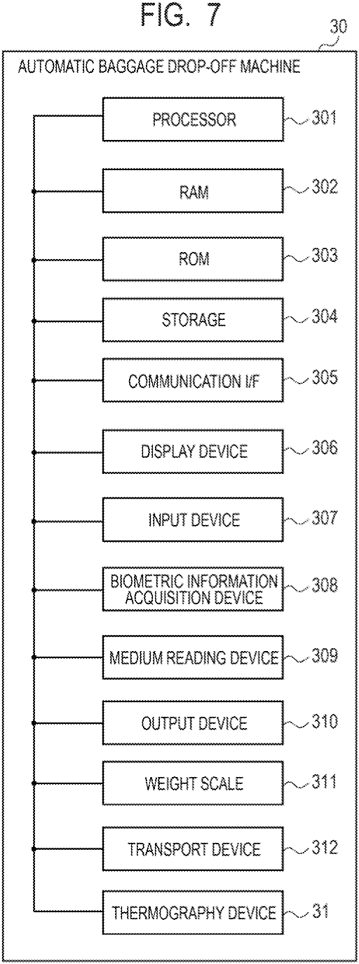

FIG. 7 is a block diagram illustrating an example of a hardware configuration of an automatic baggage drop-off machine 30 according to the present example embodiment. The automatic baggage drop-off machine 30 includes a processor 301, a RAM 302, a ROM 303, a storage 304, a communication I/F 305, a display device 306, an input device 307, a biometric information acquisition device 308, a medium reading device 309, a output device 310, a weight scale 311, a transport device 312, and the thermography device 31. These devices are connected to each other via a bus, a wiring, a drive device, or the like.

The output device 310 is a device that outputs a baggage tag attached to checked baggage. For example, the baggage tag is an RFID tag having an IC chip that stores tag information including a checked baggage ID, a token ID, a flight number, or the like. Further, the output device 310 further outputs a baggage claim tag required for claiming checked baggage after arriving at the destination. The baggage tag or the baggage claim tag is associated with at least one of a reservation number, a boarding ticket number, and a token ID, for example.

The weight scale 311 measures the weight of checked baggage and outputs a measured value to the processor 301. When the weight of checked baggage exceeds a predetermined threshold, the processor 301 outputs error information that urges the user U to take some action. The transport device 312 transports checked baggage on a receiving area placed by the user U.

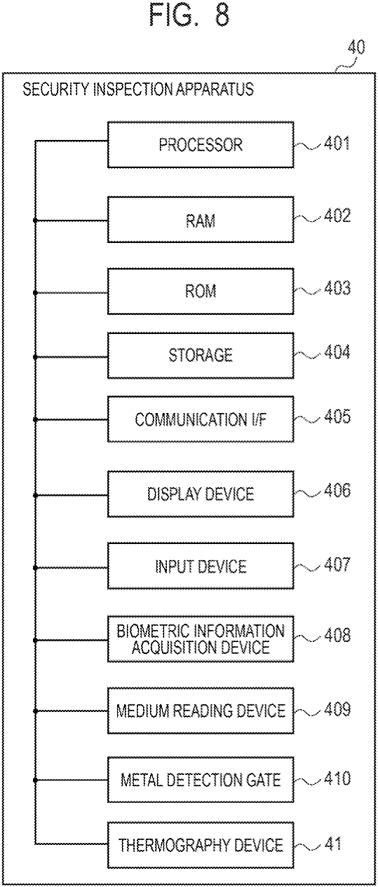

FIG. 8 is a block diagram illustrating an example of a hardware configuration of a security inspection apparatus 40 according to the present example embodiment. The security inspection apparatus 40 includes a processor 401, a RAM 402, a ROM 403, a storage 404, a communication I/F 405, a display device 406, an input device 407, a biometric information acquisition device 408, a medium reading device 409, a metal detection gate 410, and the thermography device 41. These devices are connected to each other via a bus, a wiring, a drive device, or the like.

The metal detection gate 410 is a gate-type metal detector and detects a metal item worn by a user U passing through the metal detection gate 410.

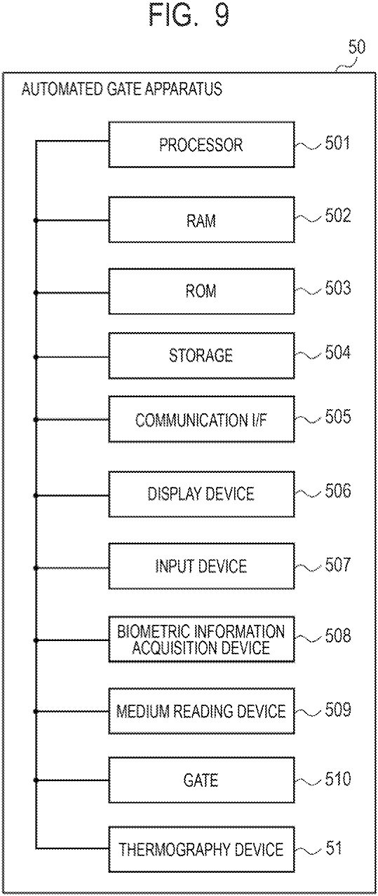

FIG. 9 is a block diagram illustrating an example of a hardware configuration of the automated gate apparatus 50 according to the present example embodiment. The automated gate apparatus 50 includes a processor 501, a RAM 502, a ROM 503, a storage 504, a communication I/F 505, a display device 506, an input device 507, a biometric information acquisition device 508, a medium reading device 509, a gate 510, and a thermography device 51. These devices are connected to each other via a bus, a wiring, a drive device, or the like.

The gate 510 transitions from a closed state to block passage of the user U during standby to an open state to permit passage of the user U under the control of the processor 501 when identity verification of the user U at the automated gate apparatus 50 is successful. The type of the gate 510 is not particularly limited and may be, for example, a flapper gate in which one or more flappers provided to one side or both sides of a passage are opened and closed, a turn style gate in which three bars are revolved, or the like.

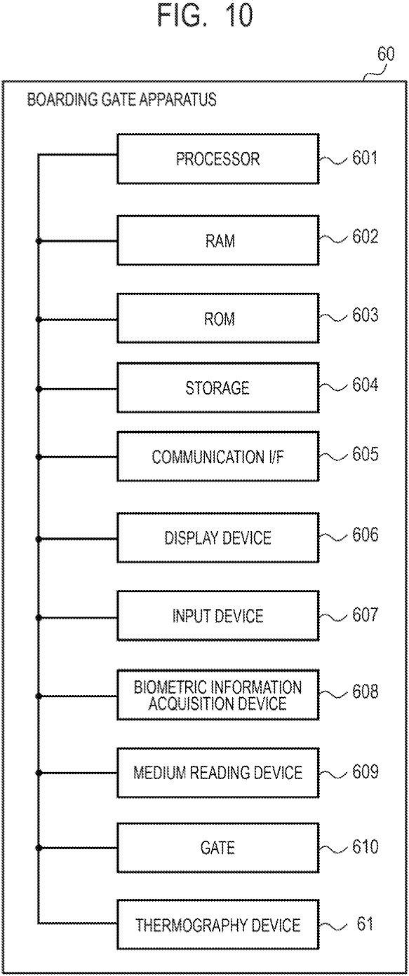

FIG. 10 is a block diagram illustrating an example of the hardware configuration of the boarding gate apparatus 60. The boarding gate apparatus 60 includes a processor 601, a RAM 602, a ROM 603, a storage 604, a communication I/F 605, a display device 606, an input device 607, a biometric information acquisition device 608, a medium reading device 609, a gate 610, and a thermography device 61. These devices are connected to each other via a bus, a wiring, a drive device, or the like.

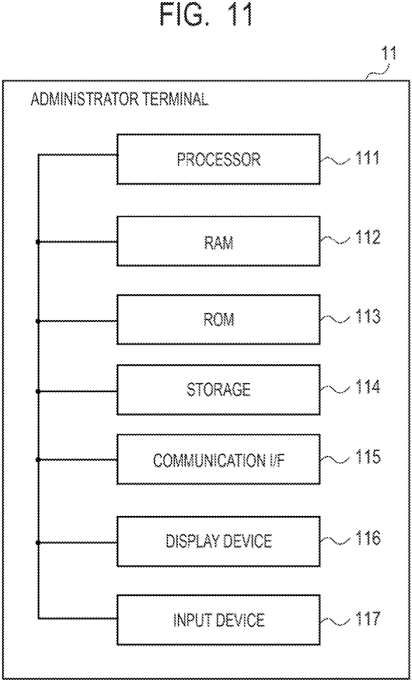

FIG. 11 is a block diagram illustrating an example of a hardware configuration of an administrator terminal 11 according to the present example embodiment. The administrator terminal 11 includes a processor 111, a RAM 112, a ROM 113, a storage 114, a communication I/F 115, a display device 116, and an input device 117. These devices are connected to each other via a bus, a wiring, a drive device, or the like.

Note that the hardware configurations illustrated in FIG. 5 to FIG. 11 are examples, a device other than the above may be added, and some of the devices may be omitted. Further, some of the devices may be replaced with another device having the same function. Further, some of the functions of the present example embodiment may be provided by another device via a network, or the functions of the present example embodiment may be distributed to and implemented by a plurality of devices. In such a way, the hardware configurations illustrated in FIG. 5 to FIG. 11 can be changed as appropriate.

Next, the operation of the apparatuses in the information processing system according to the present example embodiment will be described with reference to the drawings.

### [Check-in Procedure]

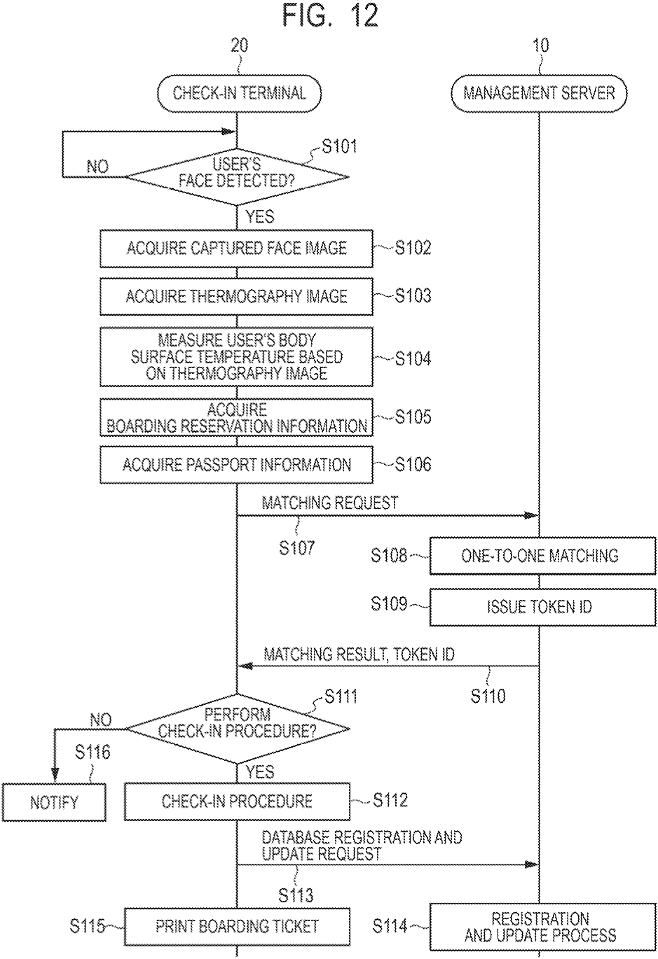

FIG. 12 is a sequence chart illustrating an example of the process in a check-in procedure of the information processing system according to the present example embodiment.

First, the check-in terminal 20 captures an image of the area in front thereof constantly or periodically and determines whether or not a face of a user U standing in front of the check-in terminal 20 is detected in the captured image (step S101). The check-in terminal 20 stands by until a face of a user U is detected in the image by the biometric information acquisition device 208 (step S101: NO).

If the check-in terminal 20 determines that a face of a user U is detected by the biometric information acquisition device 208 (step S101: YES), the check-in terminal 20 captures an image of the face of the user U and acquires the captured face image of the user U as a target face image (step S102). Note that it is preferable to display a window for obtaining user U consent before capturing a face image.

Next, the check-in terminal 20 captures an image of the face of the user U by the thermography device 21 and acquires a thermography image (step S103). That is, the check-in terminal 20 captures a thermography image in synchronization with capturing of a captured face image.

Next, the check-in terminal 20 measures the body surface temperature of the user U based on the thermography image (step S104).

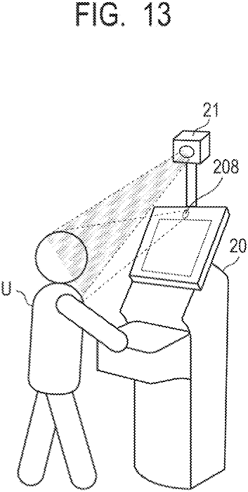

FIG. 13 is a diagram illustrating a state where a face image and a thermography image are captured at the check-in terminal 20. This illustrates an example in which a thermography image including the face of the user U is captured by the thermography device 21 while the face image of the user U is being captured by the biometric information acquisition device 208. It is preferable for the thermography device 21 to start image capturing in response to a timing of capturing performed by the biometric information acquisition device 208 rather than continuously capturing thermography images. This makes it possible to further associate thermography images with a token ID of the user U identified from the face image.

Next, in response to an airline ticket medium being held over the reading area of the medium reading device 209, the check-in terminal 20 acquires boarding reservation information on the user U from the airline ticket medium held over (step S105). The boarding reservation information includes attribute information on the user U (a family name, a first name, a gender, or the like) or flight information (an airline code, a flight number, a boarding date, a departure place, a transit point, a destination place, a seat number, a departure time, an arrival time, or the like).

Next, when a passport is held over the reading area of the medium reading device 209, the check-in terminal 20 acquires passport information on the user U from the passport held over (step S106). The passport information includes a passport face image of the user U, identity verification information, a passport number, information on a country that has issued the passport, or the like.

Next, the check-in terminal 20 requests the management server 10 to match face images (step S107). The data of the matching request includes a captured face image captured at the current place and the passport face image read from the passport.

In response to receiving information from the check-in terminal 20, the management server 10 performs one-to-one matching between the captured face image captured by the check-in terminal 20 and the passport face image (step S108).

Next, the management server 10 issues a token ID provided that the matching result in step S108 is that the matching is successful (step S109) and transmits the matching result and the token ID to the check-in terminal 20 (step S110).

Next, based on the matching result received from the management server 10, the check-in terminal 20 determines whether or not a check-in procedure for the user U is ready to be performed (step S111).

In this step, if the check-in terminal 20 determines that the check-in procedure is not ready to be performed (step S111: NO), the check-in terminal 20 notifies the user U of an error message (step S116) and ends the process.

In contrast, if the check-in terminal 20 determines that the matching result at the management server 10 is that the matching is successful and determines that the check-in procedure for the user U is ready to be performed (step S111: YES), the check-in terminal 20 performs a check-in procedure such as confirmation of an itinerary, selection of a seat, or the like based on input information from the user U (step S112). In response to completion of the check-in procedure, the check-in terminal 20 transmits a database registration and update request to the management server 10 (step S113).

Next, in response to receiving the database registration and update request from the check-in terminal 20, the management server 10 performs a registration process and an update process on the passage history information DB 10b and the operation information DB 10c (step S114). Specifically, the passage history information at the touch point TP1 and measurement history information on user U’s body surface temperature is registered to the passage history information DB 10b in association with the token ID.

The check-in terminal 20 then prints a boarding ticket describing boarding reservation information and guidance information about procedures up to boarding (step S115) and ends the process.

### [Baggage Drop-off Procedure]

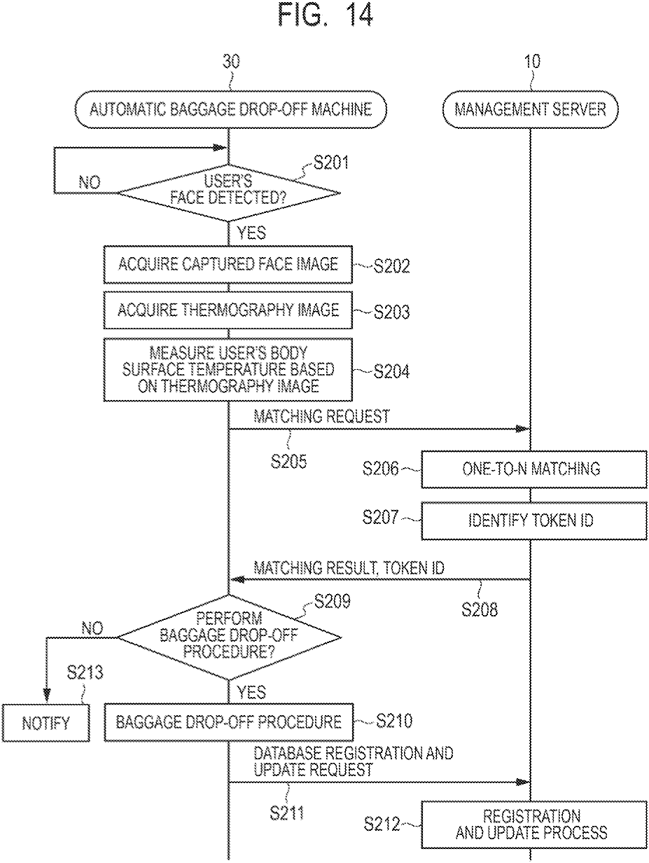

FIG. 14 is a sequence chart illustrating an example of the process in a baggage drop-off procedure of the information processing system according to the present example embodiment.

First, the automatic baggage drop-off machine 30 captures an image of the area in front of the terminal constantly or periodically and determines whether or not a face of a user U standing in front of the automatic baggage drop-off machine 30 is detected in the captured image (step S201). The automatic baggage drop-off machine 30 stands by until a face of a user U is detected in the image by the biometric information acquisition device 308 (step S201: NO).

If the automatic baggage drop-off machine 30 determines that a face of a user U is detected by the biometric information acquisition device 308 (step S201: YES), the automatic baggage drop-off machine 30 captures an image of the face of the user U and acquires the captured face image of the user U as a target face image (step S202).

Next, the automatic baggage drop-off machine 30 captures an image of the face of the user U by the thermography device 31 and acquires a thermography image (step S203). That is, the automatic baggage drop-off machine 30 captures a thermography image in synchronization with capturing of a captured face image.

Next, the automatic baggage drop-off machine 30 measures the body surface temperature of the user U based on the thermography image (step S204).

Next, the automatic baggage drop-off machine 30 requests the management server 10 to perform matching of face images (step S205). The data of the matching request includes a captured face image captured at the current place.

In response to receiving data on the matching request from the automatic baggage drop-off machine 30, the management server 10 performs one-to-N matching between the captured face image captured by the automatic baggage drop-off machine 30 and registered face images of registrants stored in the token ID information DB 10a (step S206).

Next, the management server 10 identifies the token ID of the user U provided that the matching result in step S206 is that the matching is successful (step S207).

Next, the management server 10 transmits the matching result and the token ID to the automatic baggage drop-off machine 30 (step S208). Further, to perform a baggage drop-off procedure, the management server 10 transmits operation information (for example, boarding reservation information or passport information) associated with the registered face image to the automatic baggage drop-off machine 30 together with the matching result.

Next, based on the matching result received from the management server 10, the automatic baggage drop-off machine 30 determines whether or not a baggage drop-off procedure for the user U is ready to be performed (step S209).

In this step, if the automatic baggage drop-off machine 30 determines that the matching result at the management server 10 is that the matching is unsuccessful and determines that the baggage drop-off procedure on the user is not ready to be performed (step S209: NO), the automatic baggage drop-off machine 30 notifies the user U of an error message (step S213) and ends the process.

In contrast, if the automatic baggage drop-off machine 30 determines that the matching result at the management server 10 is that the matching is successful and determines that the automatic baggage drop-off procedure for the user is ready to be performed (step S209: YES), the automatic baggage drop-off machine 30 performs the baggage drop-off procedure such as weighing of trustee baggage, issuance of baggage tags, transportation of trustee baggage (step S210).

Next, in response to the completion of the baggage drop-off procedure of the user U, the automatic baggage drop-off machine 30 transmits a database registration and update request to the management server 10 (step S211).

Then, in response to receiving the database registration and update request from the automatic baggage drop-off machine 30, the management server 10 performs a registration process and an update process on the passage history information DB 10b and the operation information DB 10c (step S212). Specifically, passage history information at the touch point TP2 and measurement history information on the body surface temperature of the user U at the touch point TP2 are registered to the passage history information DB 10b in association with the token ID.

### [Security Inspection Procedure]

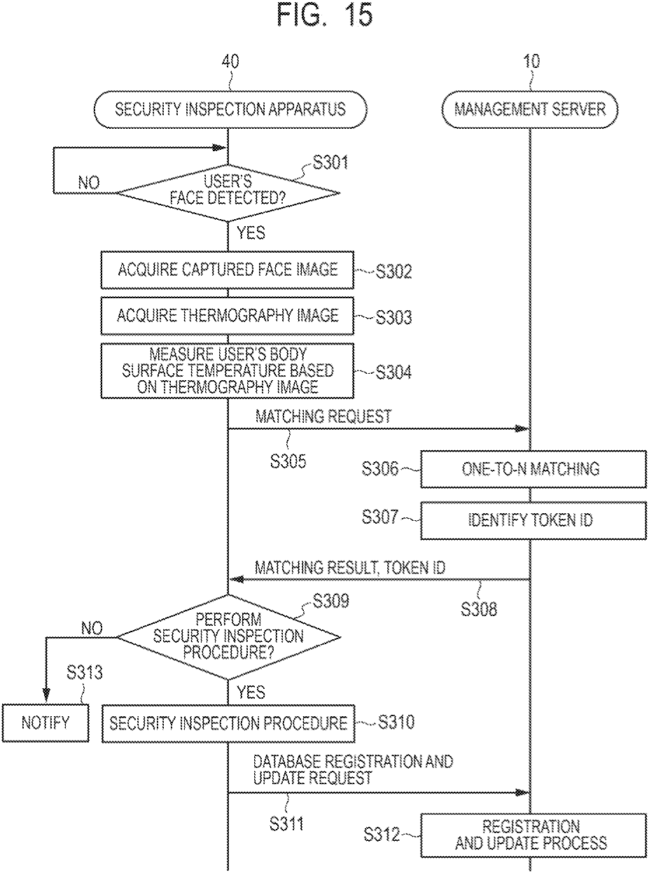

FIG. 15 is a sequence chart illustrating an example of the process in a security inspection procedure of the information processing system according to the present example embodiment.

First, the security inspection apparatus 40 captures an image of the area in front of the terminal constantly or periodically and determines whether or not a face of a user U standing in front of the security inspection apparatus 40 is detected in the captured image (step S301). The security inspection apparatus 40 stands by until a face of a user U is detected in the image by the biometric information acquisition device 408 (step S301: NO).

If the security inspection apparatus 40 determines that a face of a user U is detected by the biometric information acquisition device 408 (step S301: YES), the security inspection apparatus 40 captures an image of the face of the user U and acquires the captured face image of the user U as a target face image (step S302).

Next, the security inspection apparatus 40 captures an image of the face of the user U by the thermography device 41 and acquires a thermography image (step S303). That is, the security inspection apparatus 40 captures a thermography image in synchronization with capturing of a captured face image.

Next, the security inspection apparatus 40 measures the body surface temperature of the user U based on the thermography image (step S304).

Next, the security inspection apparatus 40 requests the management server 10 to perform matching of face images (step S305). The data of the matching request includes a captured face image captured at the current place.

In response to receiving data on the matching request from the security inspection apparatus 40, the management server 10 performs one-to-N matching between the captured face image captured by the security inspection apparatus 40 and registered face images of registrants stored in the token ID information DB 10a (step S306).

Next, the management server 10 identifies the token ID of the user U provided that the matching result in step S306 is that the matching is successful (step S307).

Next, the management server 10 transmits the matching result and the token ID to the security inspection apparatus 40 (step S308). Further, to perform a security inspection procedure, the management server 10 transmits operation information (for example, boarding reservation information or passport information) associated with the registered face image to the security inspection apparatus 40 together with the matching result.

Next, based on the matching result received from the management server 10, the security inspection apparatus 40 determines whether or not a security inspection procedure on the user U is ready to be performed (step S309).

In this step, if the security inspection apparatus 40 determines that the matching result at the management server 10 is that the matching is unsuccessful and determines that the security inspection procedure for the user is not ready to be performed (step S309: NO), the security inspection apparatus 40 notifies the user U of an error message (step S313) and ends the process.

In contrast, if the security inspection apparatus 40 determines that the matching result at the management server 10 is that the matching is successful and determines that the security inspection procedure for the user U is ready to be performed (step S309: YES), the security inspection apparatus 40 performs the security inspection procedure such as body inspection by the metal detector and baggage inspection by X-ray machine (step S310).

Next, in response to the completion of the security inspection procedure of the user U, the security inspection apparatus 40 transmits a database registration and update request to the management server 10 (step S311).

Then, in response to receiving the database registration and update request from the security inspection apparatus 40, the management server 10 performs a registration process and an update process on the passage history information DB 10b and the operation information DB 10c (step S312). Specifically, passage history information at the touch point TP3 and measurement history information on the body surface temperature of the user U at the touch point TP3 are registered to the passage history information DB 10b in association with the token ID.

### [Departure Inspection Procedure]

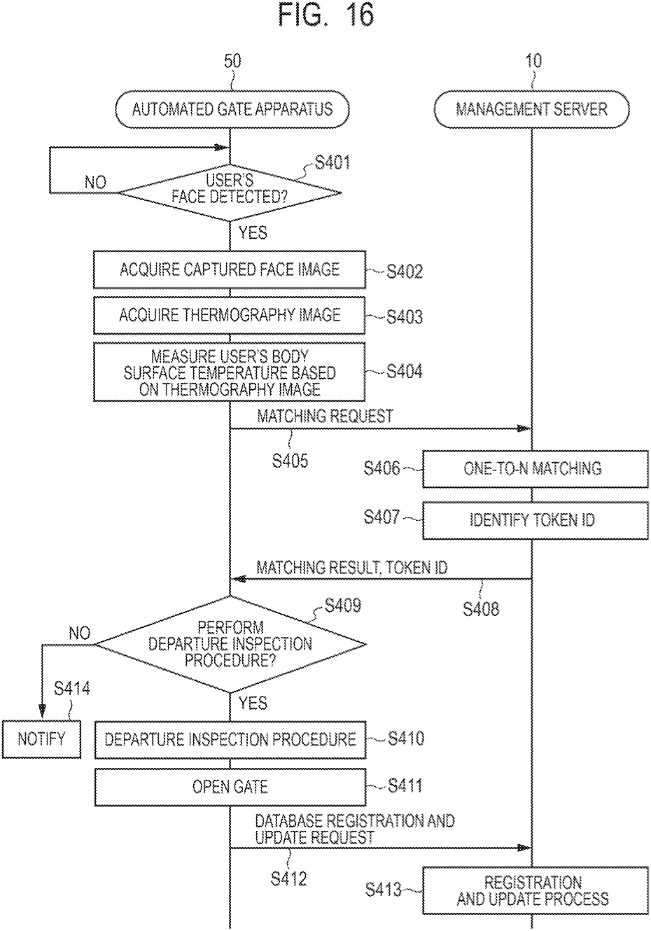

FIG. 16 is a sequence chart illustrating an example of the process in a departure inspection procedure of the information processing system according to the present example embodiment.

First, the automated gate apparatus 50 captures an image of the area in front of the terminal constantly or periodically and determines whether or not a face of a user U standing in front of the automated gate apparatus 50 is detected in the captured image (step S401). The automated gate apparatus 50 stands by until a face of a user U is detected in the image by the biometric information acquisition device 508 (step S401: NO).

If the automated gate apparatus 50 determines that a face of a user U is detected by the biometric information acquisition device 508 (step S401: YES), the automated gate apparatus 50 captures an image of the face of the user U and acquires the captured face image of the user U as a target face image (step S402).

Next, the automated gate apparatus 50 captures an image of the face of the user U by the thermography device 51 and acquires a thermography image (step S403). That is, the automated gate apparatus 50 captures a thermography image in synchronization with capturing of a captured face image.

Next, the automated gate apparatus 50 measures the body surface temperature of the user U based on the thermography image (step S404).

Next, the automated gate apparatus 50 requests the management server 10 to perform matching of face images (step S405). The data of the matching request includes a captured face image captured at the current place.

In response to receiving data on the matching request from the automated gate apparatus 50, the management server 10 performs one-to-N matching between the captured face image captured by the automated gate apparatus 50 and registered face images of registrants stored in the token ID information DB 10a (step S406).

Next, the management server 10 identifies the token ID of the user U provided that the matching result in step S406 is that the matching is successful (step S407).

Next, the management server 10 transmits the matching result and the token ID to the automated gate apparatus 50 (step S408). Further, to perform a departure inspection procedure, the management server 10 transmits operation information (for example, boarding reservation information or passport information) associated with the registered face image to the automated gate apparatus 50 together with the matching result.

Next, based on the matching result received from the management server 10, the automated gate apparatus 50 determines whether or not a departure inspection procedure for the user U is ready to be performed (step S409).

In this step, if the automated gate apparatus 50 determines that the matching result at the management server 10 is that the matching is unsuccessful and determines that the departure inspection procedure for the user is not ready to be performed (step S409: NO), the automated gate apparatus 50 notifies the user U of an error message (step S414) and ends the process.

In contrast, if the automated gate apparatus 50 determines that the matching result at the management server 10 is that the matching is successful and determines that the departure inspection procedure for the user U is ready to be performed (step S409: YES), the automated gate apparatus 50 performs the departure inspection procedure such as body inspection by the metal detector and baggage inspection by X-ray machine (step S410).

Next, if the user U is allowed to departure by the departure inspection procedure, the automated gate apparatus 50 opens the gate 510 (step S411).

Next, in response to the completion of the departure inspection procedure of the user U, the automated gate apparatus 50 transmits a database registration and update request to the management server 10 (step S412).

Then, in response to receiving the database registration and update request from the automated gate apparatus 50, the management server 10 performs a registration process and an update process on the passage history information DB 10b and the operation information DB 10c (step S413). Specifically, passage history information at the touch point TP4 and measurement history information on the body surface temperature of the user U at the touch point TP4 are registered to the passage history information DB 10b in association with the token ID.

### [Identity Verification Procedure at Boarding Gate]

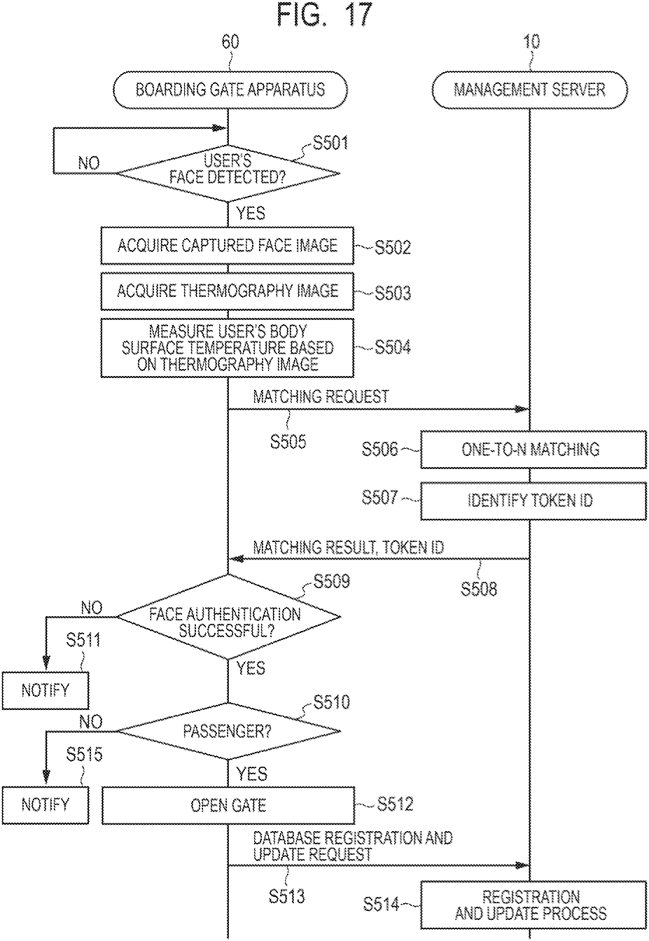

FIG. 17 is a sequence chart illustrating an example of the process in an identity verification procedure at the boarding gate of the information processing system according to the present example embodiment.

First, the boarding gate apparatus 60 captures an image of the area in front of the terminal constantly or periodically and determines whether or not a face of a user U standing in front of the boarding gate apparatus 60 is detected in the captured image (step S501). The boarding gate apparatus 60 stands by until a face of a user U is detected in the image by the biometric information acquisition device 608 (step S501: NO).

If the boarding gate apparatus 60 determines that a face of a user U is detected by the biometric information acquisition device 608 (step S501: YES), the boarding gate apparatus 60 captures an image of the face of the user U and acquires the captured face image of the user U as a target face image (step S502).

Next, the boarding gate apparatus 60 captures an image of the face of the user U by the thermography device 61 and acquires a thermography image (step S503). That is, the boarding gate apparatus 60 captures a thermography image in synchronization with capturing of a captured face image.

Next, the boarding gate apparatus 60 measures the body surface temperature of the user U based on the thermography image (step S504).

Next, the boarding gate apparatus 60 requests the management server 10 to perform the matching process of face images and the determination process of whether or not to allow boarding (step S505). The data of the matching request includes a captured face image captured at the current place.

In response to receiving data on the matching request from the boarding gate apparatus 60, the management server 10 performs one-to-N matching between the captured face image captured by the boarding gate apparatus 60 and registered face images of registrants stored in the token ID information DB 10a (step S506).

Next, the management server 10 identifies the token ID of the user U provided that the matching result in step S506 is that the matching is successful (step S507).

Next, the management server 10 transmits the matching result and the token ID to the boarding gate apparatus 60 (step S508). Further, to perform a procedure at the boarding gate, the management server 10 transmits operation information (for example, boarding reservation information or passport information) associated with the registered face image to the boarding gate apparatus 60 together with the matching result.

Next, the boarding gate apparatus 60 determines whether or not face authentication of the user U is successful at the management server 10 (step S509).

In this step, if the boarding gate apparatus 60 determines that the matching result at the management server 10 is that the matching is unsuccessful and determines that the face authentication of the user U failed (step S509: NO), the boarding gate apparatus 60 notifies the user U of an error message (step S511) and ends the process.

In contrast, if the boarding gate apparatus 60 determines that the matching result at the management server 10 is that the matching is successful and determines that the face authentication of the user U is successful (step S509: YES), the process proceeds to step S510.

In step S510, the boarding gate apparatus 60 determines whether or not the user U is a passenger of the airplane.

In this step, if the boarding gate apparatus 60 determines that the user U is not a passenger of the airplane (step S510: NO), the boarding gate apparatus 60 notifies the user U of an error message (for example, “Please check the gate number”) (step S515) and ends the process.

In contrast, the boarding gate apparatus 60 determines that the user U is a passenger of the airplane (step S510: YES), the process proceeds to step S512.

In step S512, the boarding gate apparatus 60 opens the gate 610. Accordingly, the user U passes through the boarding gate apparatus 60 and boards the airplane.

Next, in response to the completion of the identity verification procedure of the user U, the boarding gate apparatus 60 transmits a database registration and update request to the management server 10 (step S513). The data in the registration and update request includes the body surface temperature of user U measured based on the thermographic image.

Then, in response to receiving the database registration and update request from the boarding gate apparatus 60, the management server 10 performs a registration process and an update process on the passage history information DB 10b and the operation information DB 10c (step S514). Specifically, passage history information at the touch point TP5 and measurement history information on the body surface temperature of the user U at the touch point TP5 are registered to the passage history information DB 10b in association with the token ID.

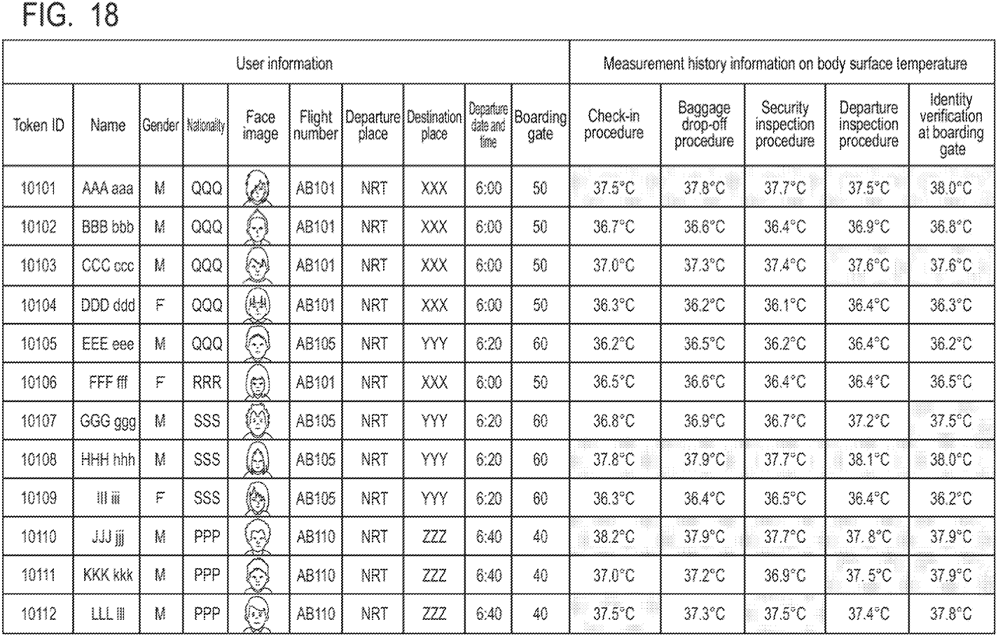

FIG. 18 is a diagram illustrating an example of measurement history information on body surface temperatures according to the present example embodiment. Here, the history information of the body surface temperature of the user U measured at the plurality of touch points is associated with the user information of the plurality of users U. Noted that the reference value of the fever state in the present example embodiment is 37.5° C.

For example, the user information of the user U whose token ID is “10101” is {Gender: “M (male)”/Nationality: “QQQ”/Departure place: “NRT”/Destination place: “XXX”/Departure time: “6: 00”/Boarding gate: “50”}. The measurement history information of the body surface temperature of the user U is {At check-in: “37.5° C.”/At baggage drop-off: “37.8° C.”/At security inspection: “37.7° C.”/At departure inspection: “37.5° C.”/At identification procedure in boarding gate: “38.0° C.”}. That is, the user U whose token ID is “10101” is a person whose body surface temperature has been measured at all touch points higher than or equal to the reference value. Therefore, in the present example embodiment, this user U is identified as a person with fever.

The measurement history information of the user U whose token ID is “10102” is {At check-in: “36.7° C.”/At baggage drop-off: “36.6° C.”/At security inspection: “36.4° C.”/At departure inspection: “36.9° C.”/At identification procedure in boarding gate: “36.8° C.”}. That is, the user U whose token ID is “10102” is a person whose body surface temperature has been measured at all touch points less than the reference value. Therefore, in the present example embodiment, this user U is not identified as a person with fever.

The measurement history information of the user U whose token ID is “10103” is {At check-in: “37.0° C.”/At baggage drop-off: “37.3° C.”/At security inspection: “37.4° C.”/At departure inspection: “37.6° C.”/At identification procedure in boarding gate: “37.6° C.”}. That is, the user U whose token ID is “10103” is a person whose body surface temperature has been measured at two points, touch point TP4 and touch point TP5, which are higher than or equal to the reference value. Therefore, in the present example embodiment, this user U is identified as a person with fever.

### [Statistical Analysis Process (1)]

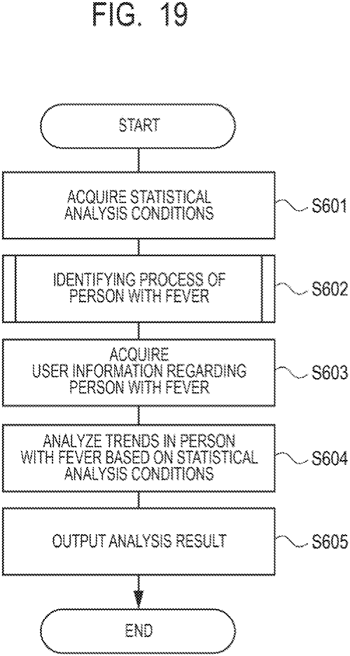

FIG. 19 is a flowchart illustrating an example of a statistical analysis process in the management server 10 according to the present example embodiment. For example, this process is performed by the management server 10 at a predetermined cycle.

First, the management server 10 acquires statistical analysis conditions stored in advance in a storage device such as a storage 104 (step S601). The statistical analysis conditions can be set in advance by an administrator or the like of the management server 10. The statistical analysis conditions include the target period for the statistical process, the unit of the period (yearly/monthly/daily), and the data items for calculating the number, percentage, and rank of persons with fever. Items such as the attribute information of user U and flight information of airplane are used as data items.

Next, the management server 10 performs an identification process for a person with fever within the target period of statistical process (step S602) to acquire a token ID related to the person with fever. Details of this process will be described later.

Next, the management server 10 refers to the operation information DB 10c using the token ID as a key to acquire user information of the person with fever (step S603).

Next, the management server 10 analyzes the trend of the person with fever based on the statistical analysis conditions (step S604). Specifically, the management server 10 calculates (A) the number of all users, (B) the number of persons with fever, (C) the number and percentage of the persons with fever for each data item such as gender, nationality, and age (generation), and (D) the percentage of the persons with fever to all users.

Then, the management server 10 outputs the analysis result to an output destination such as the storage 104 (step S605) and terminates the processing. The management server 10 may automatically output the analysis result to a server of a government agency or a company that takes measures against infectious diseases.

### [Identification Process for Person-With-Fever]

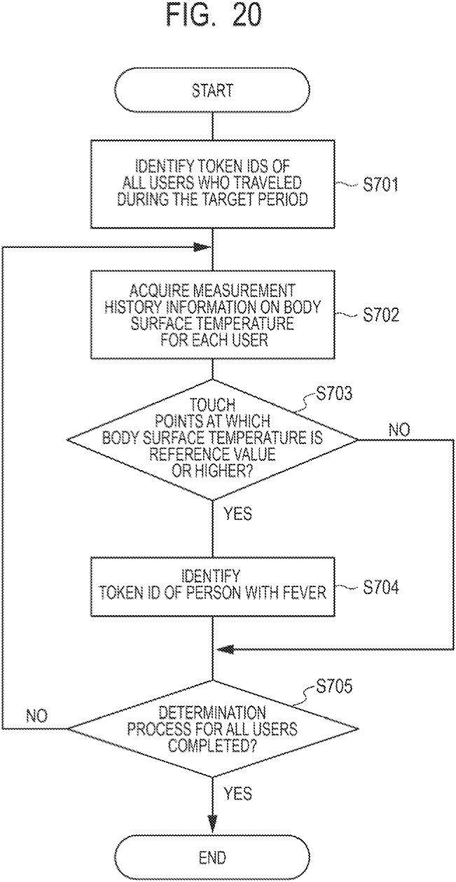

FIG. 20 is a flowchart illustrating an example of an identification process for a person with fever in the management server 10 according to the present example embodiment. This process is described in detail in step S602 of FIG. 19 above, but the method of identifying the person with fever is not limited to this.

First, the management server 10 refers to the passage history information DB 10b and identifies the token ID of all the users U who traveled during the target period (step S701).

Next, the management server 10 refers to the passage history information DB 10b and acquires the measurement history information of the body surface temperature for each user U (step S702).

Next, based on the measurement history information of the body surface temperature of the user U, the management server 10 determines whether or not there is a touch point where the body surface temperature is measured to be higher than or equal to the reference value (step S703). That is, the management server 10 determines whether or not the body surface temperature higher than or equal to the reference value is included among the plurality of body surface temperatures measured at the plurality of touch points TP1 to TP5.

In this step, if the management server 10 determines that the measurement history information of the user U includes a body surface temperature higher than or equal to the reference value (step S703: YES), the process proceeds to step S704.

In contrast, if the management server 10 determines that the measurement history information of the user U does not include a body surface temperature higher than or equal to the reference value (step S703: NO), the process proceeds to step S705.

In step S704, the management server 10 identifies the token ID of the person with fever and stores the list information of the token ID in a storage device such as the storage 104. The process then proceeds to step S705.

In step S705, the management server 10 determines whether or not the determination process has been completed for all users identified in step S701.

In this step, if the management server 10 determines that the determination process has been completed for all users (step S705: YES), the process ends.

In contrast, if the management server 10 determines that the determination process has not been completed for all users (step S705: NO), the process returns to step S702.

### [Statistical Analysis Process (2)]

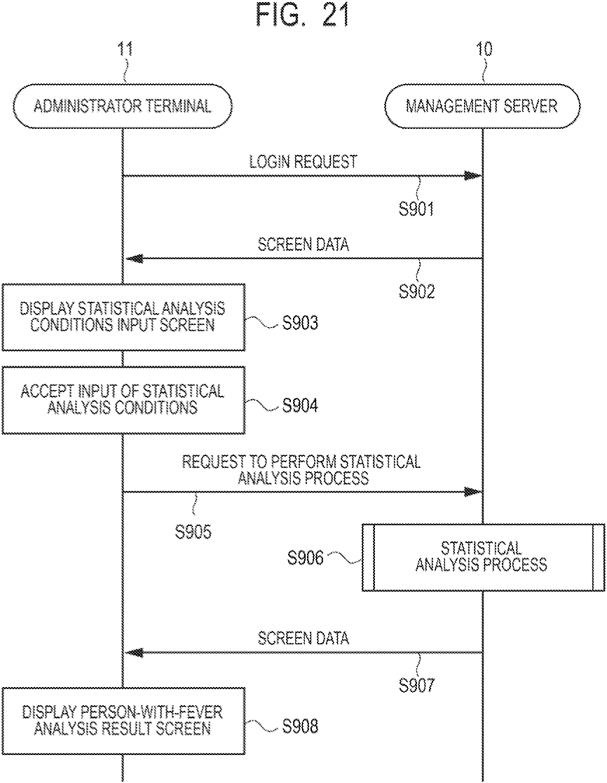

FIG. 21 is a sequence chart illustrating an example of the statistical analysis process according to the present example embodiment. This process is performed when the administrator requests the statistical analysis process using the administrator terminal 11.

First, the administrator terminal 11 makes a login request to the management server 10 (step S901). Next, if the management server 10 authenticates the administrator terminal 11 with the authentication information included in the login request, the management server 10 transmits screen data of analysis conditions input screen to the administrator terminal 11 (step S902).

Next, the administrator terminal 11 displays the statistical analysis conditions input screen on the display device 116 based on the screen data received from the management server 10 (step S903).

Next, if the administrator terminal 11 receives the input of the statistical analysis conditions from the administrator on the screen (step S904), the administrator terminal 11 requests the management server 10 to perform the statistical analysis process (step S905).

Next, the management server 10 performs the statistical analysis process based on the statistical analysis conditions received from the administrator terminal 11 (step S906). The process of step S906 is the same as that of FIG. 19 described above.

Next, the management server 10 transmits the screen data of the analysis result screen to the administrator terminal 11 as a result of the process in step S906 (step S907).

Then, when the administrator terminal 11 displays a person-with-fever analysis result screen on the display device 116 based on the screen data received from the management server 10 (step S908), the process ends.

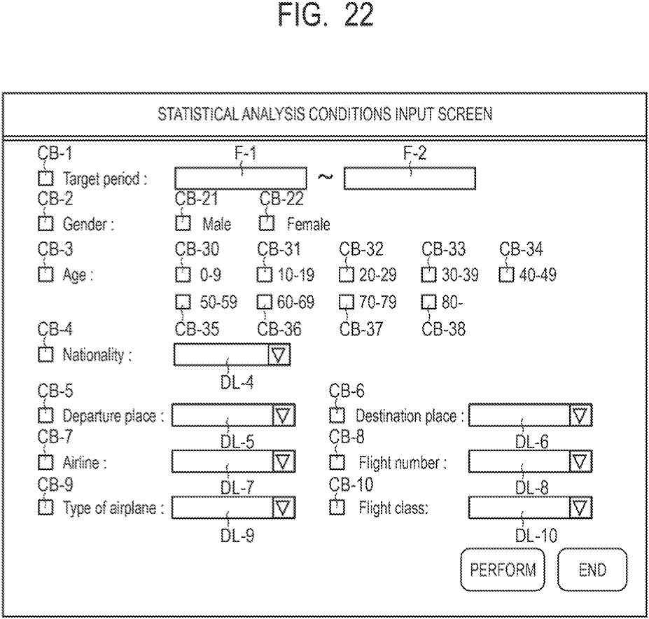

FIG. 22 is a diagram illustrating an example of a statistical analysis conditions input screen displayed on an administrator terminal 11 according to the present example embodiment. Check boxes CB-1 to CB-10 are input means for the administrator to specify whether or not to include the period covered, gender, age, nationality, departure place, destination place, airline, flight number, model of airplane, and seat class in the statistical analysis conditions. Input forms F-1 and F-2 are input means for the administrator to specify the start and end dates of the period covered. Drop down lists DL-1 to DL-7 are input means for the administrator to further specify nationality, departure place, destination place, airline, flight number, model of airplane, and seat class. Check boxes CB-21 and CB-22 are input means for the administrator to further narrow down the gender of the person with fever. Check boxes CB-30 to CB-38 are input means for the administrator to further narrow down the age of the person with fever.

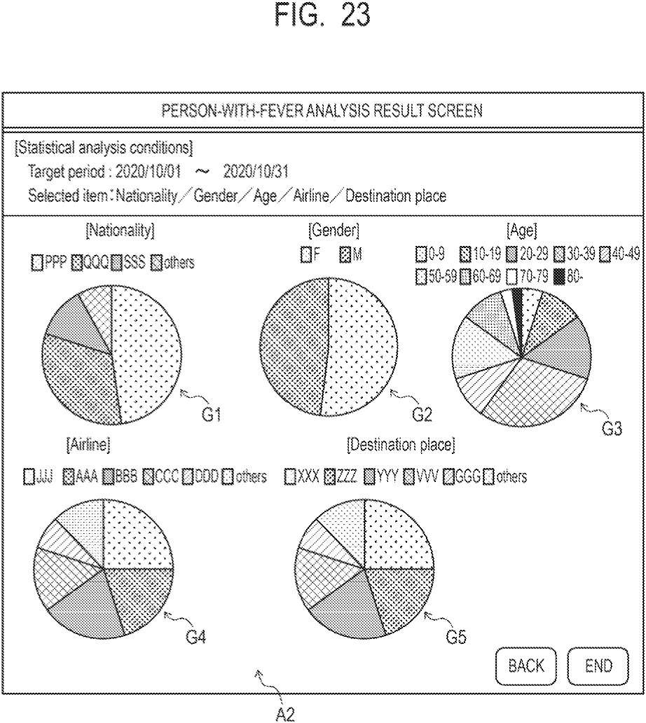

FIG. 23 is a diagram illustrating an example of the person-with-fever analysis result screen displayed on the administrator terminal 11 according to the present example embodiment. In the upper column A1 of the screen, the target period (Oct. 1, 2020 to Oct. 31, 2020) and five selection items (nationality/gender/age/airline/destination place) are displayed as statistical analysis conditions specified by the administrator.

At the bottom of the screen, in column A2, are five pie charts G1 to G5 illustrating the trends of fevers by nationality, gender, age, airline and destination as analysis results. For example, according to the pie chart G1 illustrating the trends of persons with fever by nationality, it can be seen that the number of persons with fever who have “PPP” as nationality was the highest.

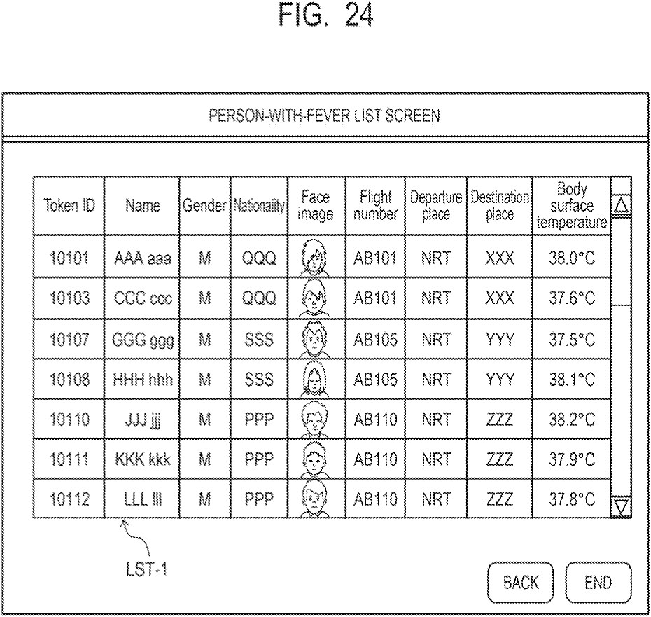

FIG. 24 is a diagram illustrating an example of a person-with-fever list screen displayed on the administrator terminal 11 according to the present example embodiment. In the center of the screen, for the user U whose body surface temperature is higher than or equal to the reference value (37.5° C.), a list LST-1 is displayed that includes data on token ID, name, gender, nationality, face image, flight number, departure place, destination place, and body surface temperature (highest value). It is preferable that the screen of FIG. 24 is displayed, for example, when the administrator designates one of the pie charts of FIG. 23 with a mouse pointer or the like.

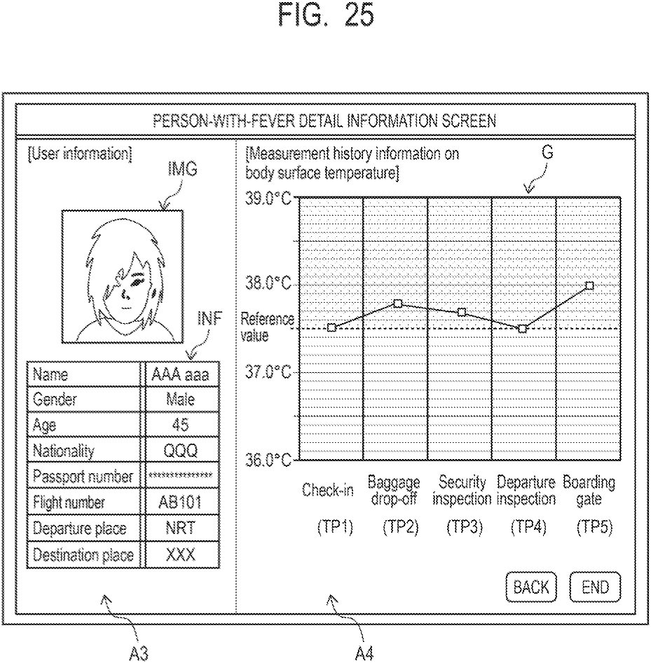

FIG. 25 is a diagram illustrating an example of a person-with-fever detail information screen displayed on the administrator terminal 11 according to the present example embodiment. In the left column A3 of the screen, in addition to a face image IMG of the person with fever, name, gender, age, nationality, passport number, flight number, departure place, and destination place are displayed as user information of the person with fever. In the right column A4 of the screen, the measurement history information of the body surface temperature at the airport DA of the person with fever is displayed by a line graph G. It is preferable that the screen of FIG. 25 is displayed, for example, when the manager designates the face image of a specific person with fever from the list in FIG. 24 with a mouse pointer or the like.

As described above, the management server 10 according to the present example embodiment identifies the persons with fever among all the users U based on the body surface temperature measured at each touch point of the airport DA, and statistically analyzes the tendency of the persons with fever based on the attribute information and flight information of the user U associated with the body surface temperature. Thus, government agencies and companies related to the prevention of infectious diseases can efficiently take measures against infectious diseases based on the analysis results. For example, government agencies can take various measures, such as restricting flights to Country A, strengthening the inspection and tracking system for people entering and leaving from Country A, providing information to government agencies of Country A, and increasing production and import and export of medical supplies, if there are many persons with fever among users U who have nationality of Country A.

### Second Example Embodiment

The information processing system in the present example embodiment will be described below. Note that references common to the references provided in the drawings in the first example embodiment represent the same components. Description of the features common to the first example embodiment will be omitted, and different features will be described in detail.

The present example embodiment differs from the first example embodiment in that the body surface temperature of the user U is measured not only at the airport DA of the first country but also at the touch point of the airport AA of the second country as subject for statistical process.

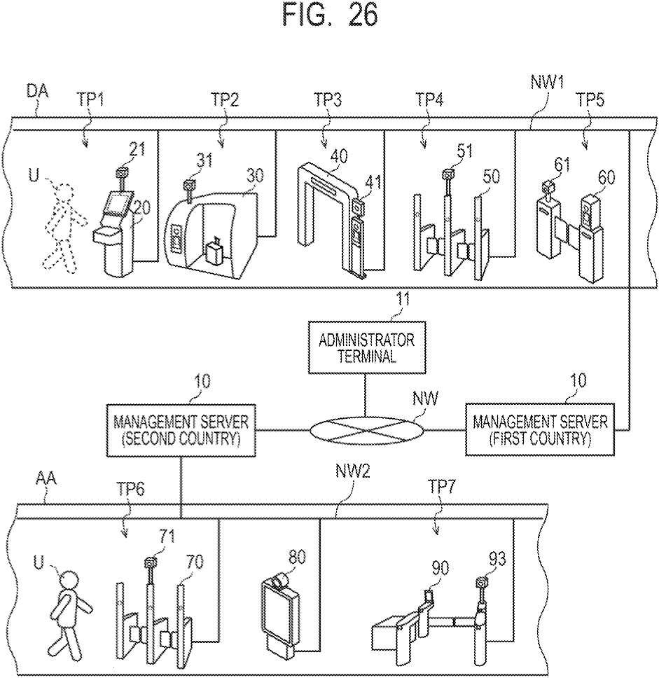

FIG. 26 is a schematic diagram illustrating an example of the overall configuration of the information processing system according to the present example embodiment. As illustrated in FIG. 26, an automated gate apparatus 70, a signage terminal 80 and an automatic customs gate apparatus 90 are installed at the airport AA. The automated gate apparatus 70, the signage terminal 80 and the automatic customs gate apparatus 90 are connected to the management server 10 of the second country via a network NW2 such as a LAN. The management server 10 of the second country is also connected to the management server 10 of the first country via a WAN such as the Internet. The management servers 10 of the first and second countries are equipped with a data coordination function.

The automated gate apparatus 70 is installed at the entry inspection site (hereafter, referred to as “touch point TP6”) in the airport AA. The automated gate apparatus 70 is an apparatus that automatically performs an entry inspection procedure on the user U. The hardware configuration of the automated gate apparatus 70 is the same as that of the automated gate apparatus 50 of the airport DA. In the present example embodiment, after completion of the entry inspection procedure at the touch point TP6, the user U moves to a customs inspection site or a quarantine inspection site.

The signage terminal 80 is installed in the airport AA. The signage terminal 80 is a display terminal for presenting, to the user U, various guidance information received from the management server 10. The signage terminal 80 of the present example embodiment is at least installed near the exit of the entry inspection site.

The automatic customs gate apparatus 90 is installed in each customs inspection site (hereafter, referred to as “touch point TP7”) in the airport AA. The automatic customs gate apparatus 90 is an electronic gate that restricts passage of the user U based on a result of face matching or the like. The user U who is permitted to pass the gate is able to exit the customs inspection site and enter the second country. The user U who is not permitted to pass the gate will be subjected to a separate examination such as being subjected to face-to-face customs inspection with staff in a manned booth (face-to-face lane), for example.

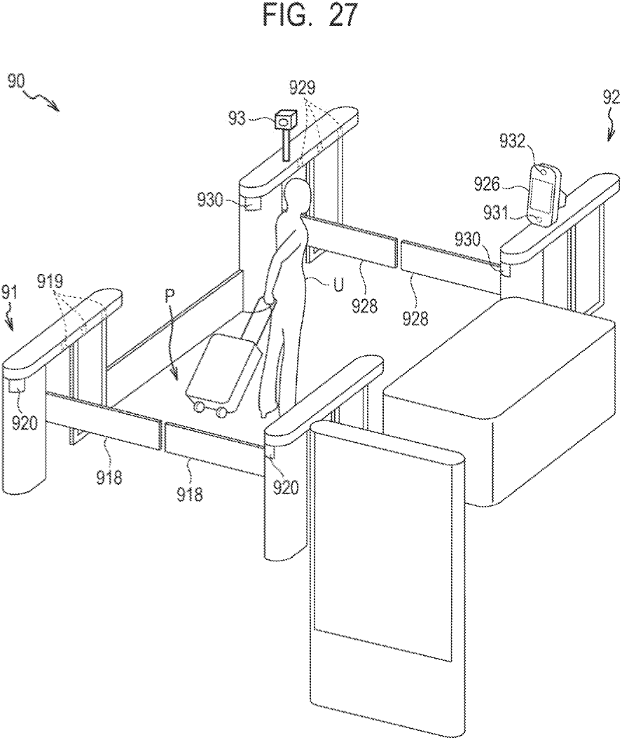

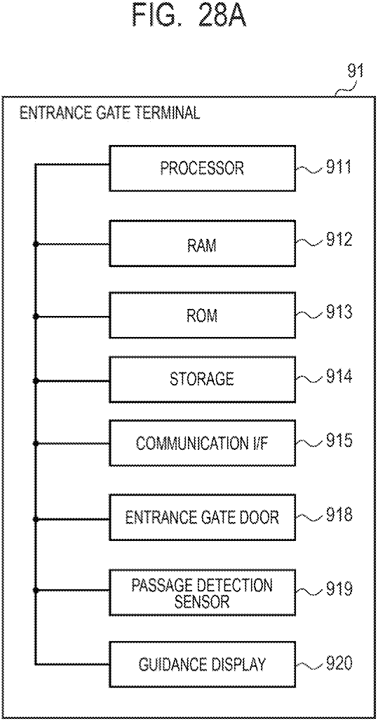

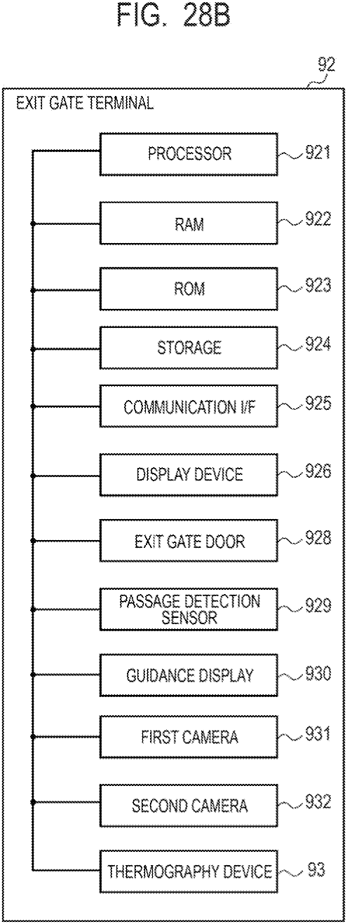

FIG. 27 is a schematic diagram illustrating an external view of an entry gate terminal 91 and an exit gate terminal 92 forming the automatic customs gate apparatus 90. FIG. 28A is a block diagram illustrating an example of a hardware configuration of the entry gate terminal 91. FIG. 28B is a block diagram illustrating an example of a hardware configuration of the exit gate terminal 92.

As illustrated in FIG. 27, the automatic customs gate apparatus 90 includes the entry gate terminal 91 and the exit gate terminal 92. The entry gate terminal 91 and the exit gate terminal 92 are installed on the entry side and on the exit side, respectively, of a gate passage P through which the user U has to pass. In the gate passage P, the user U who has entered the gate passage P is restricted from exiting a space other than the exit gate terminal 92 by a partition plate, a wall, a fence, an inspection table, or the like, for example, installed on both sides along the gate passage P.

As illustrated in FIG. 28A, the entry gate terminal 91 includes a processor 911, a RAM 912, a ROM 913, a storage 914, a communication I/F 915, an entry gate door 918, a passage detection sensor 919, and a guidance display 920. These devices are connected to each other via a bus, a wiring, a drive device, or the like.

The entry gate door 918 is an open/close door that performs a door opening operation and a door closing operation under the control of the processor 911 and transitions between a door opened state that permits passage of the user U and a door closed state that blocks passage of the user U. The opening/closing type of the entry gate door 918 is not particularly limited and may be, for example, a flapper type, a slide type, a revolving type, or the like.

In response to detecting passage of the user U, the passage detection sensor 919 outputs an output signal indicating the passage of the user U. The processor 911 can determine whether or not the user U has passed through the entry gate terminal 91 and entered the gate passage P based on the output signals from a plurality of passage detection sensors 919 and the output order thereof.

Each guidance display 920 displays display indicating whether or not to permit entry to the gate passage under the control of the processor 911. When the entry gate door 918 is in an open state, the guidance display 920 displays that entry to the gate passage is permitted. Further, when the entry gate door 918 is in a closed state, the guidance display 920 displays that entry to the gate passage is not allowed. The guidance display 920 can display whether or not to permit entry to the gate passage P by color display, symbol display, text display, or the like, for example.

As illustrated in FIG. 28B, the exit gate terminal 92 includes a processor 921, a RAM 922, a ROM 923, a storage 924, a communication I/F 925, a display device 926, an exit gate door 928, a passage detection sensor 929, a guidance display 930, a first camera 931, a second camera 932, and a thermography device 93. These devices are connected to each other via a bus, a wiring, a drive device, or the like.

The exit gate door 928 is an open/close door that performs a door opening operation and a door closing operation under the control of the processor 921 and transitions between a door closed state that blocks passage of the user U and a door opened state that permits passage of the user U.

The first camera 931 is a long-range camera that has an image-capturing range including at least the inside of the gate passage P and is able to capture an image of a more distant area than the second camera 932. The second camera 932 is a short-range camera having an image-capturing range including at least the area in front of the exit gate terminal 92. Note that the positions at which the first camera 931 and the second camera 932 are provided are not particularly limited and can be any position where respective image-capturing ranges can be achieved.

### [Data Coordination Process Between Two Countries]

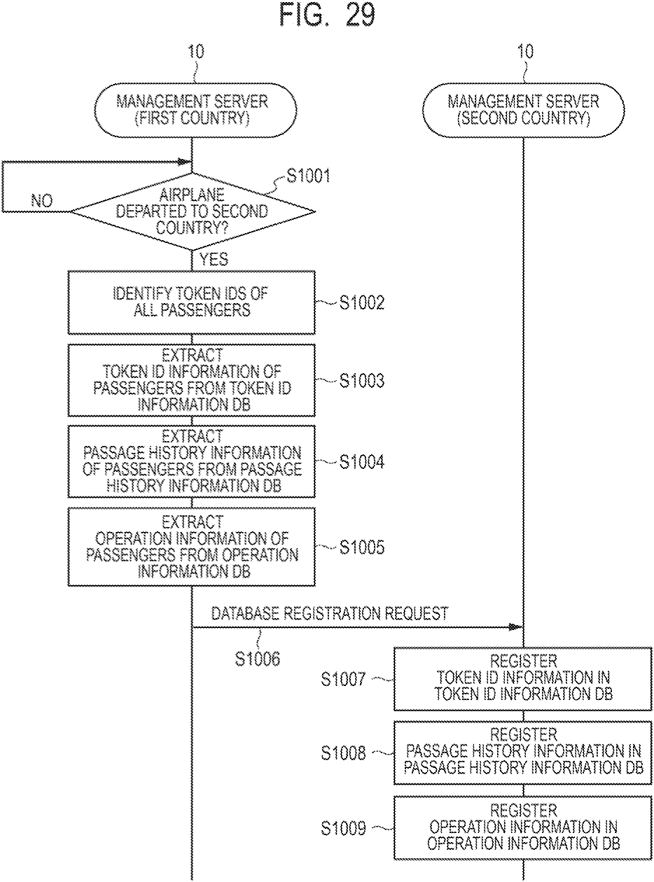

FIG. 29 is a sequence chart illustrating an example of a data coordination process between two countries in the information processing system according to the present example embodiment. This process is performed after an airplane takes off from the airport DA of the first country and before the airplane arrives at the airport AA of the second country, for example.

First, the management server 10 of the first country determines whether or not the airplane has departed to the second country (step S1001). In this step, if the management server 10 of the first country determines that the airplane has departed from the first country to the second country (step S1001: YES), the process proceeds to step S1002.

In contrast, if the management server 10 of the first country determines that the airplane has not yet departed from the first country to the second country (step S1001: NO), the process of step S1001 is repeated.

In step S1002, the management server 10 of the first country identifies token IDs from the passage history information DB 10b for the passengers of the airplane that has departed to the second country.

Next, the management server 10 of the first country extracts token ID information on the passengers from the token ID information DB 10a by using token IDs as keys (step S1003).

Next, the management server 10 of the first country extracts passage history information on the passengers from the passage history information DB 10b by using token IDs as keys (step S1004).

Next, the management server 10 of the first country extracts operation information on the passengers from the operation information DB 10c by using token IDs as keys (step S1005).

Next, the management server 10 of the first country transmits the token ID information, the passage history information, and the operation information extracted for passengers to the management server 10 of the second country and requests for database registration (step S1006).

Next, the management server 10 of the second country registers the token ID information received from the management server 10 of the first country to the token ID information DB 10a (step S1007).

Next, the management server 10 of the second country registers the passage history information received from the management server 10 of the first country to the passage history information DB 10b (step S1008).

The management server 10 of the second country then registers the operation information received from the management server 10 of the first country to the operation information DB 10c (step S1009) and ends the process. Accordingly, data related to the passengers are shared between the management server 10 of the first country and the management server 10 of the second country. That is, the measurements history information on body surface temperature in the first country will be available for persons entering the second country.

### [Entry Inspection Procedure]

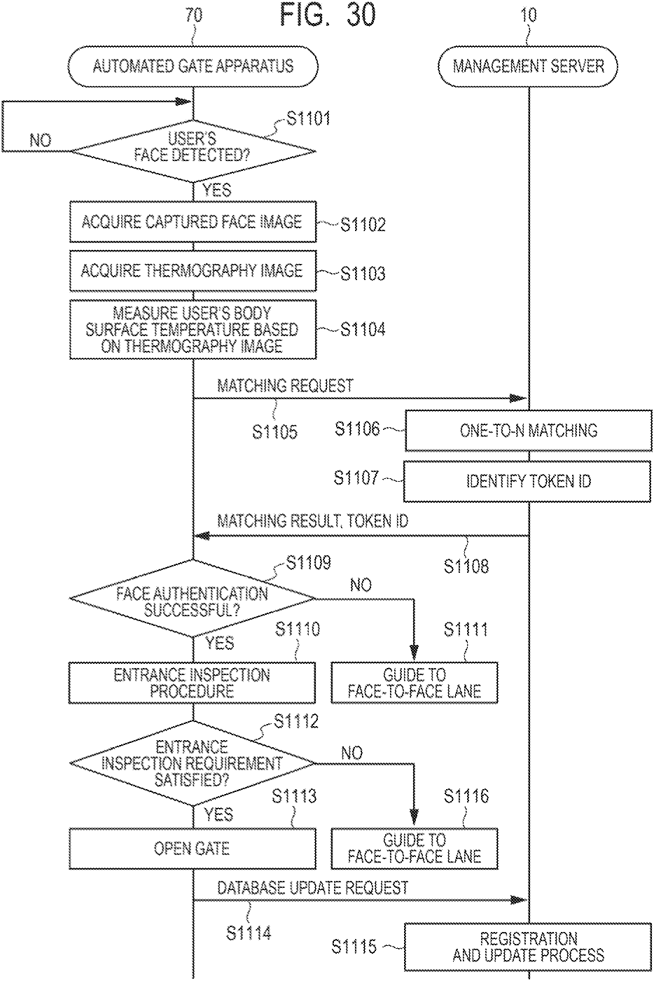

FIG. 30 is a sequence chart illustrating an example of a process in an entry inspection procedure performed by the information processing system according to the present example embodiment.

First, the automated gate apparatus 70 captures an image of the area in front of the terminal constantly or periodically and determines whether or not a face of a user U standing in front of the automated gate apparatus 70 is detected in the captured image (step S1101). The automated gate apparatus 70 stands by until a face of a user U is detected in the image by the biometric information acquisition device 708 (step S1101: NO).

If the automated gate apparatus 70 determines that a face of a user U is detected by the biometric information acquisition device 708 (step S1101: YES), the automated gate apparatus 70 captures an image of the face of the user U and acquires the captured face image of the user U as a target face image (step S1102).

Next, the automated gate apparatus 70 captures the face of the user U by the thermography device 71 and acquires a thermography image (step S1103). That is, the automated gate apparatus 70 captures a thermography image in synchronization with capturing of the captured face image.

Next, the automated gate apparatus 70 measures the body surface temperature of the user U based on the thermography image (step S1104).

Next, the automated gate apparatus 70 requests the management server 10 to perform matching of face images and determination of an inspection target (step S1105). The data of the matching request includes a captured face image captured at the current place.

In response to receiving data on the matching request from the automated gate apparatus 70, the management server 10 performs one-to-N matching between the captured face image captured by the automated gate apparatus 70 and registered face images of registrants stored in the token ID information DB 10a (step S1106).

Next, the management server 10 identifies the token ID of the user U provided that the matching result in step S1106 is that the matching is successful (step S1107).

Next, the management server 10 transmits the matching result and the token ID to the automated gate apparatus 70 (step S1108). Further, to perform the entry inspection procedure, the management server 10 transmits operation information (for example, boarding reservation information or passport information) associated with the registered face image to the automated gate apparatus 70 together with the matching result.

Next, based on the matching result received from the management server 10, the automated gate apparatus 70 determines whether or not the entry inspection procedure for the user U is ready to be performed (step S1109).

In this step, if the automated gate apparatus 70 determines that the matching result at the management server 10 is that the matching is unsuccessful and thus determines that the entry inspection procedure for the user U is not ready to be performed (step S1109: NO), the automated gate apparatus 70 displays a guidance message to guide the user U to a face-to-face lane (step S1111) and ends the process.

In contrast, if the automated gate apparatus 70 determines that the matching result at the management server 10 is that the matching is successful and thus determines that the entry inspection procedure for the user U is ready to be performed (step S1109: YES), the automated gate apparatus 70 performs the entry inspection procedure (step S1110). The process then proceeds to step S1112.

In step S1112, the automated gate apparatus 70 determines whether or not the user U is the user U is a person who satisfies requirements of entry inspection. In this step, if the automated gate apparatus 70 determines that the user U is a person who satisfies the requirements of the entry inspection (step S1112: YES), the process then proceeds to step S1113.

In contrast, if the automated gate apparatus 70 determines that the user U is not a person who satisfies the requirements of the entry inspection (step S1112: NO), the automated gate apparatus 70 displays a guidance message to guide the user U to a face-to-face lane (step S1116) and ends the process.

In step S1113, the automated gate apparatus 70 opens the gate 710 when the entry of the user U is permitted by the entry inspection procedure.

Next, in response to the completion of the entry inspection procedure, the automated gate apparatus 70 transmits a database registration and update request to the management server 10 (step S1114).

Then, in response to receiving the database registration and update request from the automated gate apparatus 70, the management server 10 performs a registration process and an update process on the passage history information DB 10b and the operation information DB 10c (step S1115). Specifically, passage history information at the touch point TP6 and measurement history information on the body surface temperature of the user U at the touch point TP6 are registered to the passage history information DB 10b in association with the token ID.

### [Customs Inspection Procedure]

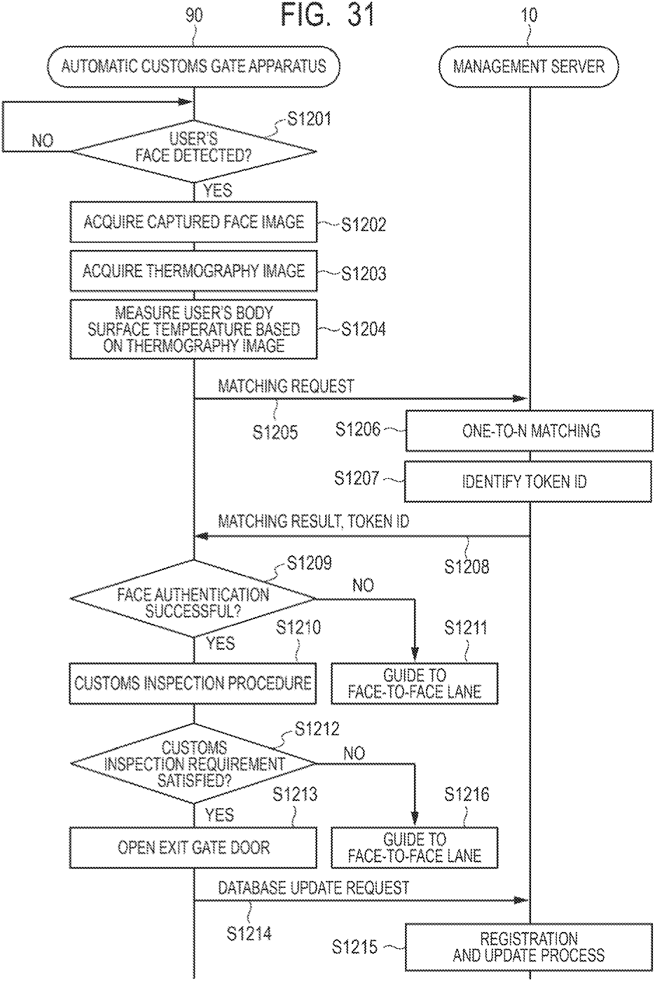

FIG. 31 is a sequence chart illustrating an example of a process in a customs inspection procedure performed by the information processing system according to the present example embodiment.

First, the automatic customs gate apparatus 90 captures an image of the area in front of the terminal constantly or periodically and determines whether or not a face of a user U standing in front of the automatic customs gate apparatus 90 is detected in the captured image (step S1201). The automatic customs gate apparatus 90 stands by until a face of a user U is detected in the image by the first camera 931 and the second camera 932 (step S1201: NO).

If the automatic customs gate apparatus 90 determines that a face of a user U is detected by the first camera 931 or the second camera 932 (step S1201: YES), the automatic customs gate apparatus 90 captures an image of the face of the user U and acquires the captured face image of the user U as a target face image (step S1202).

Next, the automatic customs gate apparatus 90 captures the face of the user U by the thermography device 93 and acquires a thermography image (step S1203). That is, the automatic customs gate apparatus 90 captures a thermography image in synchronization with capturing of the captured face image.

Next, the automatic customs gate apparatus 90 measures the body surface temperature of the user U based on the thermography image (step S1204).

Next, the automatic customs gate apparatus 90 requests the management server 10 to perform matching of face images and determination of a target for quarantine inspection (step S1205). The data of the matching request includes a captured face image captured at the current place.

In response to receiving data on the matching request from the automatic customs gate apparatus 90, the management server 10 performs one-to-N matching between the captured face image captured by the automatic customs gate apparatus 90 and registered face images of registrants stored in the token ID information DB 10a (step S1206).

Next, the management server 10 identifies the token ID of the user U provided that the matching result in step S1206 is that the matching is successful (step S1207).

Next, the management server 10 transmits the matching result and token ID to the automatic customs gate apparatus 90(step S1208). Further, to perform the customs inspection procedure, the management server 10 transmits operation information (for example, boarding reservation information or passport information) associated with the registered face image to the automatic customs gate apparatus 90 together with the matching result.

Next, based on the matching result received from the management server 10, the automatic customs gate apparatus 90 determines whether or not the customs inspection procedure for the user U is ready to be performed (step S1209).

In this step, if the automatic customs gate apparatus 90 determines that the matching result at the management server 10 is that the matching is unsuccessful and thus determines that the customs inspection procedure for the user U is not ready to be performed (step S1209: NO), the automatic customs gate apparatus 90 displays a guidance message to guide the user U to a face-to-face lane (step S1211) and ends the process.

In contrast, if the automatic customs gate apparatus 90 determines that the matching result at the management server 10 is that the matching is successful and thus determines that the customs inspection procedure for the user U is ready to be performed (step S1209: YES), the automatic customs gate apparatus 90 performs the customs inspection (step S1210). The process then proceeds to step S1212.

In step S1212, the automatic customs gate apparatus 90 determines whether or not the user U is a person who satisfies requirements of customs inspection. In this step, if the automatic customs gate apparatus 90 determines that the user U is a person who satisfies the requirements of the customs inspection (step S1212: YES), the process proceeds to step S1213.

In contrast, if the automatic customs gate apparatus 90 determines that the user U is not a person who satisfies the requirements of the customs inspection (step S1212: NO), the automatic customs gate apparatus 90 displays a guidance message to guide the user U to a face-to-face lane (step S1216) and ends the process.

In step S1213, the automatic customs gate apparatus 90 opens the exit gate door 928 when the entry of the user U is permitted by the customs inspection procedure.

Next, in response to the completion of the customs inspection procedure, the automatic customs gate apparatus 90 transmits a database registration and update request to the management server 10 (step S1214).

Then, in response to receiving the database registration and update request from the automatic customs gate apparatus 90, the management server 10 performs a registration process and an update process on the passage history information DB 10b and the operation information DB 10c (step S1215). Specifically, passage history information at the touch point TP7 and measurement history information on the body surface temperature of the user U at the touch point TP7 are registered to the passage history information DB 10b in association with the token ID.

As described above, according to the present example embodiment, in addition to the effect of the first example embodiment, the management server 10 has the effect of performing statistical process of the person with fever for three patterns of information: (1) measurement history information of body surface temperature measured in the departure airport, (2) measurement history information of body surface temperature measured in the arrival airport, and (3) measurement history information of body surface temperature measured at all touch points from the departure airport to the arrival airport. This also makes it possible to detect, for example, where the user U was at the departure airport, the airplane, and the arrival airport when he/she developed a fever.

### Third Example Embodiment

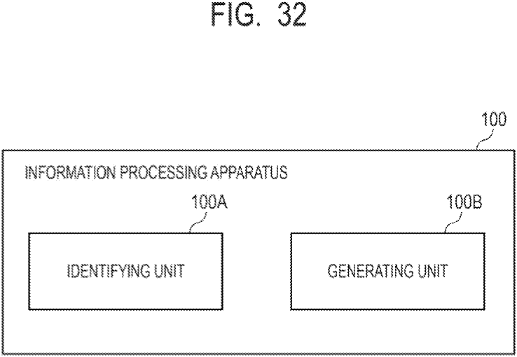

FIG. 32 is a function block diagram of an information processing apparatus 100 according to the present example embodiment. The information processing apparatus 100 includes an identifying unit 100A and a generating unit 100B. The identifying unit 100A identifies a person with fever at an airport. The generating unit 100B generates statistical information related to the person with fever based on user information acquired from the person with fever.

According to the present example embodiment, the information processing apparatus 100 that can detect the overall fever situation regarding airport users U.

### Modified Example Embodiment

Although the present invention has been described above with reference to the example embodiments, the present invention is not limited to the example embodiments described above. Various modifications that can be understood by those skilled in the art can be made to the configuration and details of the present invention within the scope not departing from the spirit of the present invention. For example, it should be understood that an example embodiment in which a configuration of a part of any of the example embodiments is added to another example embodiment or an example embodiment in which a configuration of a part of any of the example embodiments is replaced with a configuration of a part of another example embodiment is also an example embodiment to which the present invention may be applied.

The scope of each of the example embodiments also includes a processing method that stores, in a storage medium, a program that causes the configuration of each of the example embodiments to operate so as to implement the function of each of the example embodiments described above, reads the program stored in the storage medium as a code, and executes the program in a computer. That is, the scope of each of the example embodiments also includes a computer readable storage medium. Further, each of the example embodiments includes not only the storage medium in which the program described above is stored but also the individual program itself.

As the storage medium, for example, a floppy (registered trademark) disk, a hard disk, an optical disk, a magneto-optical disk, a CD-ROM, a magnetic tape, a nonvolatile memory card, or the like can be used. Further, the scope of each of the example embodiments also includes an example that operates on OS to perform a process in cooperation with another software or a function of an add-in board without being limited to an example that performs a process by an individual program stored in the storage medium.

The whole or part of the example embodiments disclosed above can be described as, but not limited to, the following supplementary notes.

### (Supplementary Note 1)

An information processing apparatus comprising:
an identifying unit that identifies a person with fever at an airport; and
a generating unit that generates statistical information related to the person with fever based on user information acquired from the person with fever.

### (Supplementary Note 2)

The information processing apparatus according to supplementary note 1, wherein the user information includes at least one of as following:

gender, age, and nationality of the person with fever, a flight number, a departure place, and an arrival place related to an aircraft on which the person with fever boarded.

### (Supplementary Note 3)

The information processing apparatus according to supplementary note 1 or 2, further comprising:
an acquisition unit that acquires biometric information and the user information of a user acquired at a procedure place in the airport, and acquires a body surface temperature of the user measured at the procedure place;
a matching unit that performs a matching process between the biometric information and registered biometric information of the user registered in advance; and
a processing unit that associates the registered biometric information, the body surface temperature, and the user information when the user is successfully authenticated by the matching process.

### (Supplementary Note 4)

The information processing apparatus according to supplementary note 3, wherein the identifying unit identifies the person with fever based on the body surface temperature.

### (Supplementary Note 5)

The information processing apparatus according to supplementary note 3 or 4, wherein the identifying unit identifies the person with fever among a plurality of users based on the measurement history information of the body surface temperature measured at each of a plurality of procedure places.

### (Supplementary Note 6)

The information processing apparatus according to supplementary note 5, wherein the identifying unit identifies the user as the person with fever when there are a predetermined number of values equal to or larger than a reference value among a plurality of body surface temperatures included in the measurement history information of the user.

### (Supplementary Note 7)

The information processing apparatus according to supplementary note 5, wherein the identifying unit identifies the user as the person with fever when the body surface temperature at least measured at the predetermined procedure place is equal to or higher than the reference value among a plurality of the body surface temperatures included in the measurement history information of the user.

### (Supplementary Note 8)

The information processing apparatus according to any one of supplementary notes 1 to 7, wherein the generating unit automatically generates the statistical information based on pre-specified conditions.

### (Supplementary Note 9)

The information processing apparatus according to any one of supplementary notes 1 to 7, wherein the generating unit generates the statistical information based on conditions specified in an external terminal and outputs the statistical information to the external terminal.

### (Supplementary Note 10)

An information processing method comprising:
identifying a person with fever at an airport; and
generating statistical information related to the person with fever based on user information acquired from the person with fever.

### (Supplementary Note 11)

A storage medium storing a program that causes a computer to perform:
identifying a person with fever at an airport; and
generating statistical information related to the person with fever based on user information acquired from the person with fever.

Reference Signs List

| | |
|---|---|
| NW | network |
| 10 | management server |
| 10a | token ID information DB |
| 10b | passage history information DB |
| 10c | operation information DB |
| 11 | administrator terminal |
| 20 | check-in terminal |
| 30 | automatic baggage drop-off machine |
| 40 | security inspection apparatus |
| 50,70 | automated gate apparatus |
| 60 | boarding gate apparatus |
| 80 | signage terminal |
| 90 | automatic customs gate apparatus |
| 91 | entry gate terminal |
| 92 | exit gate terminal |
| 100 | information processing apparatus |
| 100A | identifying unit |
| 100B | generating unit |

What is claimed is:

1] An information processing apparatus comprising:
at least one memory storing instructions; and
at least one processor configured to execute the instructions to:
identify a person with fever at an airport; and
generate statistical information related to the person with fever based on user information acquired from the person with fever.

2] The information processing apparatus according to claim 1, wherein the user information includes at least one of as following:
gender, age, and nationality of the person with fever, a flight number, a departure place, and an arrival place related to an aircraft on which the person with fever boarded.

3] The information processing apparatus according to claim 1, wherein the at least one processor is further configured to execute the instructions to:
acquire biometric information and the user information of a user acquired at a procedure place in the airport, and acquires a body surface temperature of the user measured at the procedure place;
perform a matching process between the biometric information and registered biometric information of the user registered in advance; and
associate the registered biometric information, the body surface temperature, and the user information when the user is successfully authenticated by the matching process.

4] The information processing apparatus according to claim 3, wherein the at least one processor identifies the person with fever based on the body surface temperature.

5] The information processing apparatus according to claim 3, wherein the at least one processor identifies the person with fever among a plurality of users based on the measurement history information of the body surface temperature measured at each of a plurality of procedure places.

6] The information processing apparatus according to claim 5, wherein the at least one processor identifies the user as the person with fever when there are a predetermined number of values equal to or larger than a reference value among a plurality of body surface temperatures included in the measurement history information of the user.

7] The information processing apparatus according to claim 5, wherein the at least one processor identifies the user as the person with fever when the body surface temperature at least measured at the predetermined procedure place is equal to or higher than the reference value among a plurality of the body surface temperatures included in the measurement history information of the user.

8] The information processing apparatus according to claim 1, wherein the at least one processor automatically generates the statistical information based on pre-specified conditions.

9] The information processing apparatus according to claim 1, wherein the generating at least one processor generates the statistical information based on conditions specified in an external terminal and outputs the statistical information to the external terminal.

10] An information processing method comprising:
identifying a person with fever at an airport; and
generating statistical information related to the person with fever based on user information acquired from the person with fever.

11] A non-transitory storage medium storing a program that causes a computer to perform:
identifying a person with fever at an airport; and
generating statistical information related to the person with fever based on user information acquired from the person with fever.
